(19) United States
(12) Patent Application Publication
(10) Pub. No.: US 2009/0325944 A1
(43) Pub. Date: Dec. 31, 2009

(54) Methods and Compositions for Modulating Glycosylation

(76) Inventors: Suzanne Walker Kahne, Brookline, MA (US); Benjamin Gross, Gladwyne, PA (US)

Correspondence Address: Harvard University & Medical School;c/o Wolf, Greenfield & Sacks, P.C., 600 Atlantic Avenue, Boston, MA, 02210-2206, US

(21) Appl. No.: 12/226,151

(22) PCT Filed: Apr. 11, 2007
(86) PCT No.: PCT/US2007/008806

§ 371 Date: Sep. 11, 2009

Related U.S. Application Data
(60) Provisional application No. 60/791,535, filed on Apr. 12, 2006.

(51) Int. Cl. A61K 31/54; C07D 279/06; C07D 263/58; C07D 409/04; C07D 215/227; C07D 239/22; C07D 235/26; C07D 471/04; A61K 31/423; A61K 31/4725; A61K 31/47; A61K 31/513; A61K 31/4184; A61K 31/519; A61K 31/4436; C07H 21/04; C07K 14/00; C12Q 1/68; A61P 3/00; A61P 9/00

(52) U.S. Cl. 514/227.2; 544/54; 548/221; 546/141; 546/157; 544/297; 548/306.4; 544/250; 546/280.4; 514/376; 514/309; 514/312; 514/275; 514/395; 514/267; 514/336; 536/23.1; 530/350; 435/6

(57) ABSTRACT

The invention relates to methods and products for modulating glycosylation of proteins. The invention is useful for treating glycosylation-associated disorders such as neurodegeneration, diabetes, including complications of diabetes such as insulin resistance, nephropathy, microvascular damage, and endothelial dysfunction. The invention also relates in part to assays that are useful for identifying and testing candidate compounds for modulating glycosylation of proteins.

Aspects of the invention may have been made using finding from the National Institutes of Health grants AI44854 and 1RO3 MH076518-01. Accordingly, the United States Government may have rights in the invention.

### FIELD OF THE INVENTION

The invention relates to methods and products for modulating glycosylation of proteins. The invention is useful for treating glycosylation-associated disorders such as neurodegeneration, insulin resistance, diabetes, and complications of diabetes such as nephropathy, microvascular damage, and endothelial dysfunction. The invention also relates in part to assays that are useful for identifying and testing candidate compounds for modulating glycosylation of proteins.

### BACKGROUND OF THE INVENTION

The hexosamine biosynthetic pathway (HSP) is a minor branch of the glycolytic pathway, diverting 3-5% of cellular glucose toward the synthesis of UDP-GlcNAc, which is either transported to the golgi and used in the synthesis of complex glycans or remains in the cytoplasm where it is the obligatory substrate for O-GlcNAc Transferase (OGT). OGT is the sole known enzyme to catalyze the transient glycosylation of serine and threonine residues on many nuclear and cytoplasmic proteins (termed O-GlcNAcylation). This post-translational modification is dynamic and is a general method, like protein phosphorylation, of signal transduction.

Excess flux through the HSP has been implicated in both early (insulin resistance) and late (nephropathy, microvascular damage) stages in the course of diabetes mellitus, both in vivo and in vitro. Diabetes involves a deficiency in the availability and/or utilization of insulin. Insulin is a hormone produced by the pancreas and is necessary for cells to utilize glucose. Insulin resistance is a condition in which muscle, fat, and liver cells do not use insulin properly. As a result, the pancreas produces more insulin, which also cannot be properly used. Eventually, the pancreas cannot keep up with the body's need for insulin, and excess glucose builds up in the bloodstream. Thus, in insulin resistance, there may be high levels of blood glucose and high levels of insulin circulating in the bloodstream at the same time.

Experiments have shown that insulin resistance due to increased hexosamine flux is caused by hyper O-GlcNAcylation. Diabetics have increased production of two adipokines directly responsible for vascular injury, plasminogen activator inhibitor-1 (PAI-1) and transforming growth factor β₁(TGF-β1). Transcription of both of these proteins is decreased in cell culture when levels of O-GlcNAcylation were decreased. The molecular mechanism for this is known; increased transcription is mediated by the O-GlcNAcylation state of the transcription factor Sp1.

OGT activity and levels of O-GlcNAcylation have also been implicated in other disease states, such as Alzheimer's disease and cancer, though the role of OGT activity in these diseases has not been well studied.

### SUMMARY OF THE INVENTION

The invention relates in part to newly identified compounds that inhibit O-Glc-NAc transferase (OGT) activity. We have identified a number of compounds that inhibit O-GlcNAcylation by OGT. O-GlcNAcylation is the transient glycosylation of serine and/or threonine residues on nuclear and cytoplasmic proteins that is catalyzed by OGT. The newly identified compounds and analogs, derivatives, and variants thereof may be useful for the treatment (including active and/or prophylactic treatment) of diseases and disorders associated with hyper O-GlcNAcylation. The invention includes, in part, methods for treating diseases and conditions resulting from abnormal O-GlcNAcylation and compositions for treating such diseases and conditions. In addition, we have identified methods of assaying candidate compounds for the ability to modulate O-GlcNAcylation.

The assay also relates, in some aspects, to assays that are useful to identify compounds (e.g., small molecules, etc) that inhibit OGT activity.

According to one aspect of the invention, isolated compounds are provided. The compounds include compounds set forth as compound 4, or a derivative, analog, or variant thereof; compound 5, or a derivative, analog, or variant thereof; compound 6, or a derivative, analog, or variant thereof; compound 7, or a derivative, analog, or variant thereof; compound 8, or a derivative, analog, or variant thereof; compound 9, or a derivative, analog, or variant thereof; compound 10, or a derivative, analog, or variant thereof; compound 11, or a derivative, analog, or variant thereof; compound 12, or a derivative, analog, or variant thereof; compound 13, or a derivative, analog, or variant thereof; or compound 14, or a derivative, analog, or variant thereof.

According to another aspect of the invention, compositions are provided. The compositions include any of the aforementioned compounds or the invention. In some embodiments, the compositions also include a pharmaceutically acceptable carrier.

According to yet another aspect of the invention, methods for treating an OGT-associated disease or condition in a subject are provided. The methods include administering to a subject in need of such treatment an effective amount of an OGT-inhibiting compound to treat the OGT-associated disease or condition. In some embodiments, the OGT-inhibiting compound is the compound set forth as compound 4 or an analog, derivative, or variant thereof that inhibits OGT activity. In certain embodiments, the OGT-inhibiting compound is the compound set forth as compound 5 or an analog, derivative, or variant thereof that inhibits OGT activity. In some embodiments, the OGT-inhibiting compound is the compound set forth as compound 6 or an analog, derivative, or variant thereof that inhibits OGT activity. In some embodiments, the OGT-inhibiting compound is the compound set forth as compound 7 or an analog, derivative, or variant thereof that inhibits OGT activity. In some embodiments, the OGT-inhibiting compound is the compound set forth as compound 8 or an analog, derivative, or variant thereof that inhibits OGT activity. In certain embodiments, the OGT-inhibiting compound is the compound set forth as compound 9 or an analog, derivative, or variant thereof that inhibits OGT activity. In some embodiments, the OGT-inhibiting compound is the compound set forth as compound 10 or an analog, derivative, or variant thereof that inhibits OGT activity. In some embodiments, the OGT-inhibiting compound is the compound set forth as compound 11 or an analog, derivative, or variant thereof that inhibits OGT activity. In some embodiments, the OGT-inhibiting compound is the compound set forth as compound 12 or an analog, derivative, or variant thereof that inhibits OGT activity. In certain embodiments, the OGT-inhibiting compound is the compound set forth as compound 13 or an analog, derivative, or variant thereof that inhibits OGT activity. In some embodiments, the OGT-inhibiting compound is the compound set forth as compound 14 or an analog, derivative, or variant thereof that inhibits OGT activity. In some embodiments, the subject is human. In some embodiments, the OGT-inhibiting compound is linked to a targeting molecule. In some embodiments, the OGT-inhibiting compound is administered prophylactically to a subject at risk of having a OGT-associated disease or disorder. In certain embodiments, the OGT-inhibiting compound is administered in combination with an additional drug for treating a OGT-associated disease or disorder. In some embodiments, the OGT-associated disease or disorder is Alzheimer's disease; cancer; diabetes mellitus, insulin resistance, or a complication of diabetes. In some embodiments, the complication of diabetes is microvascular damage, insulin resistance, vascular damage, nephropathy, skin ulcers, circulatory damage, diabetic nephropathy, diabetic retinopathy, macro-vascular disease, micro-vascular disease, or diabetic neuropathy.

According to another aspect of the invention, methods of evaluating the effect of a candidate compound on OGT activity are provided. The methods include contacting OGT bound to a probe with a candidate compound and determining the displacement of the probe from the OGT, wherein the displacement of the probe from the OGT indicates that the candidate compound inhibits OGT activity. In some embodiments, the probe is compound 2 or 3. In some embodiments, the displacement of the probe from the OGT is determined using a method comprising detecting fluorescence polarization.

According to yet another aspect of the invention, kits for treating a subject in accordance with any of the aforementioned methods are provided. The kits include a package housing a first container containing at least one dose of an OGT-inhibiting compound, and instructions for using the OGT-inhibiting compound in the treatment of an OGT-associated disease or disorder. In certain embodiments, the OGT-inhibiting compound is the compound set forth as compound 4 or an analog, derivative, or variant thereof that inhibits OGT activity. In some embodiments, the OGT-inhibiting compound is the compound set forth as compound 5 or an analog, derivative, or variant thereof that inhibits OGT activity. In some embodiments, the OGT-inhibiting compound is the compound set forth as compound 6 or an analog, derivative, or variant thereof that inhibits OGT activity. In some embodiments, the OGT-inhibiting compound is the compound set forth as compound 7 or an analog, derivative, or variant thereof that inhibits OGT activity. In certain embodiments, the OGT-inhibiting compound is the compound set forth as compound 8 or an analog, derivative, or variant thereof that inhibits OGT activity. In some embodiments, the OGT-inhibiting compound is the compound set forth as compound 9 or an analog, derivative, or variant thereof that inhibits OGT activity. In some embodiments, the OGT-inhibiting compound is the compound set forth as compound 10 or an analog, derivative, or variant thereof that inhibits OGT activity. In certain embodiments, the OGT-inhibiting compound is the compound set forth as compound 11 or an analog, derivative, or variant thereof that inhibits OGT activity. In some embodiments, the OGT-inhibiting compound is the compound set forth as compound 12 or an analog, derivative, or variant thereof that inhibits OGT activity. In some embodiments, the OGT-inhibiting compound is the compound set forth as compound 13 or an analog, derivative, or variant thereof that inhibits OGT activity. In some embodiments, the OGT-inhibiting compound is the compound set forth as compound 14 or an analog, derivative, or variant thereof that inhibits OGT activity. In certain embodiments, the OGT-inhibiting compound is linked to a targeting molecule. In some embodiments, the OGT-inhibiting compound is administered prophylactically to a subject at risk of having a OGT-associated disease or disorder. In some embodiments, the OGT-inhibiting compound is administered in combination with an additional drug for treating a OGT-associated disease or disorder. In some embodiments, the OGT-associated disease or disorder is Alzheimer's disease; cancer, diabetes mellitus, insulin resistance, or a complication of diabetes. In some embodiments, the complication of diabetes is microvascular damage, insulin resistance, vascular damage, nephropathy, skin ulcers, circulatory damage, diabetic nephropathy, diabetic retinopathy, macro-vascular disease, micro-vascular disease, or diabetic neuropathy.

According to yet another aspect of the invention, methods for inhibiting OGT activity in a cell or tissue are provided. The methods include contacting the cell or tissue with an effective amount of an OGT-inhibiting compound to inhibit OGT activity in the cell, or tissue. In certain embodiments, the OGT-inhibiting compound is the compound set forth as compound 4 or an analog, derivative, or variant thereof that inhibits OGT activity. In some embodiments, the OGT-inhibiting compound is the compound set forth as compound 5 or an analog, derivative, or variant thereof that inhibits OGT activity. In some embodiments, the OGT-inhibiting compound is the compound set forth as compound 6 or an analog, derivative, or variant thereof that inhibits OGT activity. In some embodiments, the OGT-inhibiting compound is the compound set forth as compound 7 or an analog, derivative, or variant thereof that inhibits OGT activity. In some embodiments, the OGT-inhibiting compound is the compound set forth as compound 8 or an analog, derivative, or variant thereof that inhibits OGT activity. In certain embodiments, the OGT-inhibiting compound is the compound set forth as compound 9 or an analog, derivative, or variant thereof that inhibits OGT activity. In some embodiments, the OGT-inhibiting compound is the compound set forth as compound 10 or an analog, derivative, or variant thereof that inhibits OGT activity. In some embodiments, the OGT-inhibiting compound is the compound set forth as compound 11 or an analog, derivative, or variant thereof that inhibits OGT activity. In certain embodiments, the OGT-inhibiting compound is the compound set forth as compound 12 or an analog, derivative, or variant thereof that inhibits OGT activity. In some embodiments, the OGT-inhibiting compound is the compound set forth as compound 13 or an analog, derivative, or variant thereof that inhibits OGT activity. In some embodiments, the OGT-inhibiting compound is the compound set forth as compound 14 or an analog, derivative, or variant thereof that inhibits OGT activity. In certain embodiments, the OGT-inhibiting compound is linked to a targeting molecule.

According to yet another aspect of the invention, isolated nucleic acid molecules are provided. The isolated nucleic acid molecule includes the nucleotide sequence set forth as SEQ ID NO: 1.

According to another aspect of the invention an isolated protein encoded by the nucleotide sequence set forth as SEQ ID NO:1 is provided.

According to yet another aspect of the invention, an isolated polypeptide having the amino acid sequence set forth as SEQ ID NO:2 is provided. The isolated polypeptide in some embodiments is used in an assay to assess inhibitors of OGT activity.

According to some aspects of the invention, methods for confirming the effect of a candidate compound as an inhibitor of OGT activity are provided. The methods include (a) contacting a sample containing the candidate compound with (i) an OGT substrate polypeptide, (ii) a UDP-GlcNAc probe comprising detectably labeled GlcNAc, and (iii) OGT; and (b) determining the amount of transfer of the labeled GlcNAc to the OGT substrate polypeptide in the sample, wherein a lower amount of GlcNAc transferred to the OGT substrate in the sample compared to a control amount of GlcNAc transfer to the OGT substrate confirms the effect of the candidate compound as an inhibitor of OGT activity. In some embodiments, the OGT substrate polypeptide comprises an amino acid sequence that binds to a filter and/or an amino acid that can be visualized by UV detection methods. In some embodiments, the amino acid sequence that binds to the filter comprises the sequence KKK. In some embodiments, the amino acid that is visualized by UV detection methods is Y. In some embodiments, the OGT substrate polypeptide comprises the amino acid sequence set forth as SEQ IS NO:2. In some embodiments, the control amount of GlcNAc transfer to the OGT substrate is the amount of GlcNAc transfer to the OGT substrate in the absence of the candidate compound. In some embodiments, the OGT is sOGT, mOGT, or ncOGT. In some embodiments, the UDP-GlcNAc probe comprising detectably labeled GlcNAc is UDP-¹⁴GlcNAc. In some embodiments, wherein means for determining comprises blotting the contacted sample of step (a) onto a filter, washing the filter, and measuring the amount of detectably labeled OGT substrate polypeptide retained on the filter as a measure of the amount of transfer of GlcNAc to the OGT substrate polypeptide. In some embodiments, the filter is a phosphocellulose filter. In some embodiments of the invention, methods of evaluating the effect of a candidate compound on OGT activity also include confirming the effect of the candidate compound on OGT activity using any embodiment of the aforementioned aspect of the invention.

In some aspects, the invention includes the use of the foregoing compositions in the preparation of a medicament, particularly a medicament for treatment of a disease or condition associated with abnormal O-GlcNAcylation.

In some aspects of the invention, analogs, derivatives, or variants of a core sequence set forth herein as compound 27 or compound 28 may be used as OGT inhibitors in methods of the invention. Exemplary derivatives and/or variants are provided herein as compounds 15-26.

These and other objects of the invention will be described in further detail in connection with the detailed description of the invention.

### BRIEF DESCRIPTION OF THE DRAWINGS

FIG. 1 shows schematic diagram of OGT constructs and PAGE blot of the constructs. FIG. 1A provides diagrams of OGT constructs expressed in E. coli. The columns represent α-helices, two of which comprise a TPR. ncOGT and sOGT are identical to known splice variants; ΔmOGT is 50 residues shorter than mOGT (ΔmOGT is SEQ ID NO:6). FIG. 1B shows a PAGE gel of these three constructs after IMAC purification.

FIG. 2 shows a plot and two tables of results of analysis of kinetics of ncOGT and sOGT with nup62. FIG. 2A shows the kinetics of ncOGT and sOGT with nup62. The reaction was run for 18 minutes and the ¹⁴C-GlcNAc used has a specific activity of 2.72×10⁻⁶ nmole/dpm. FIG. 2B provides kinetics parameters of ncOGT and sOGT with nup62 and a peptide substrate.

FIG. 3 shows an IC₅₀ curve of compound 5 with both sOGT and ncOGT.

FIG. 4 shows a non-linear (FIG. 4A) and a linear (FIG. 4B) regression analysis of compound 6 with sOGT and as a function of UDP-¹⁴C-GlcNAc concentration.

FIG. 5 provides a schematic diagram of synthesis of UDP-GlcNAc analogs (probes).

FIG. 6 provides plot of H (hits) versus FP (false positives), demonstrating distinction of hits from false positives due to compounds with inherent fluorescence emission at 535 n.

FIG. 7 shows anisotropy of probe 2 and probe 3 mixed with a range of sOGT concentrations.

FIG. 8 shows two graphs showing displacement of 50 nM probe 3 pre-equilibrated with 3 μM sOGT concentrations by UDP-GlcNAc (FIG. 8A) and UDP (FIG. 8B)

FIG. 9 shows chemical structures of donor analogue displacement probes used in the assay.

FIG. 10 shows three of the validated OGT inhibitors identified with the high throughput screen.

FIG. 11 shows diagrams and sequences of the sOGT splice variants The underlined portion of MOGT is the proposed mitochondrial signal sequence. FIG. 11 is adapted from Hanover, J. A. et al., Arch Biochem Biophys, 1003 409(2):287-97.

### DETAILED DESCRIPTION OF THE INVENTION

The methods of the invention involve the administration of compounds that modulate the activity of O-Glc-NAc transferase (OGT) in the transient glycosylation of serine/threonine residues on proteins such as nuclear or cytoplasmic proteins (O-GlcNAcylation). OGT catalyzes the transfer of the N-acetylglucosamine from the activated sugar donor uridine diphosphate N-acetylglucosamine (UDP-GlcNAc) to a serine or threonine residue in a protein, e.g. to a protein or polypeptide substrate. As used herein, the terms “protein” and “polypeptide” are used interchangeably. Compositions of the invention include compounds that modulate (e.g. inhibit) OGT activity in cells, tissues, and subjects. As used herein, the term “OGT-inhibiting compounds” means compounds that reduce OGT glycosylation of serine and/or threonine residues of proteins. The methods of the invention, in some aspects, involve the administration of an OGT-inhibiting compound and are useful to reduce or prevent cell death and/or damage or disease that is associated with hyper O-GlcNAcylation. The invention, in some aspects, also includes the use of OGT splice variants.

As used herein, the terms “O-GlcNAcylation-associated disease or disorder” and “OGT-associated disease or disorder” include, but are not limited to diseases and disorders in which there is abnormal OGT activity and/or abnormal levels of O-GlcNAcylation. As used herein, the term “OGT activity” means OGT-mediated O-GlcNAcylation. An abnormal level of OGT activity and/or O-GlcNAcylation may be a level that is higher than a normal level or may be a level that is lower than a normal level, wherein a “normal” level is the level in a subject who does not have a disease or disorder associated with OGT activity or O-GlcNAcylation. Examples of diseases and disorders associated with OGT activity and/or O-GlcNAcylation levels include, but are not limited to neurodegenerative disorders such as Alzheimer's disease; cancer; diabetes mellitus, insulin resistance, and complications of diabetes or other OGT-associated diseases. As used herein, the term “complication of diabetes” is used to mean a disorder that is associated with diabetes. Non-limiting examples of complications of diabetes include microvascular damage, insulin resistance, vascular damage, nephropathy, skin ulcers, circulatory damage, diabetic nephropathy, diabetic retinopathy, macro-vascular disease, micro-vascular disease, and diabetic neuropathy. Thus, in some aspects of the invention, an OGT-inhibiting compound may be used to treat a subject with diabetes.

The term “diabetic” as used herein, means a subject who, at the time the sample is taken, has a primary deficiency of insulin. The term diabetic includes, but is not limited to, individuals with juvenile diabetes (Type I diabetes), adult-onset diabetes (Type 2 diabetes), gestational diabetes, and any other conditions of insulin deficiency and/or abnormally high levels of OGT activity and/or O-GlcNAcylation. The terms “diabetic” and “diabetes” are terms of art, known and understood by those practicing in the medical profession, a formal definition of which can be found in Harrison's Principles of Medicine (Harrisons, Vol 14, Principles of Internal Medicine, Eds. Fauci, A. S., E. Braunwald, K. J. Isselbacher, J. D. Wilson, J. B. Martin, D. L. Kasper, S. L. Hauser, D. L. Longo, McGraw-Hill, New York, 1999).

Subjects with blood glucose levels that are higher than normal but not yet in the range associated with a diagnosis of diabetes may be considered to have “pre-diabetes.” Pre-diabetes is also known in the art as “impaired fasting glucose” (IFG) or “impaired glucose tolerance” (IGT). Subjects with pre-diabetes have a higher risk of developing type 2 diabetes, which is also known as adult-onset diabetes or noninsulin-dependent diabetes. Subjects with pre-diabetes frequently go on to develop type 2 diabetes within 10 years, without intervention—such as diet change and/or activity changes. Health effects associated with diabetes may include heart attack, stroke, blindness, deafness, amputations, kidney failure, burning foot syndrome, venous insufficiency with ulceration and stasis dermatitis. Subjects with pre-diabetes also have a higher risk of heart disease. Insulin resistance can also occur in people who have type 1 diabetes, especially if they are overweight.

It has been discovered that the deleterious effects seen in these diseases and/or disorders that are triggered by abnormal OGT activity may be ameliorated by the administration of compounds and/or compositions that modulate OGT activity. The compounds of the invention include compounds that modulate OGT activity in the O-GlcNAcylation of proteins in cells and/or tissues, thereby reducing the cell and tissue damage and clinical manifestations of an OGT-associated disease or disorder. In some embodiments of the invention, the compounds inhibit OGT activity.

Table 1 provides examples of OGT-inhibiting compounds of the invention.

TABLE 1
OGT-inhibiting compounds.
Compounds 4-6

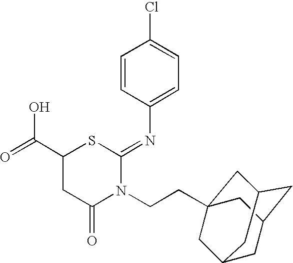

4

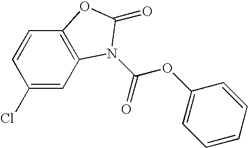

5

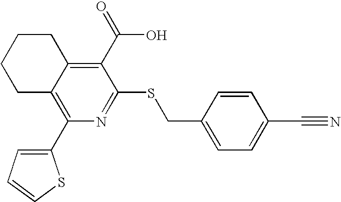

6
Compounds 7-14

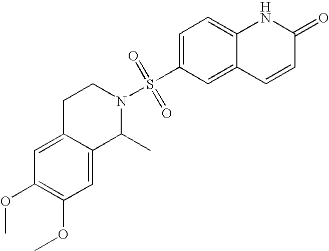

7

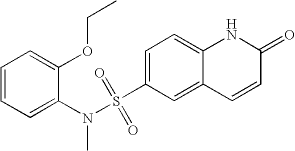

8

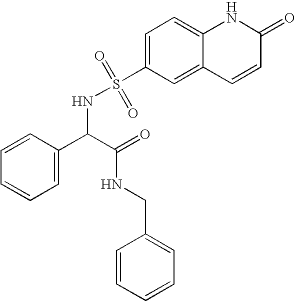

9

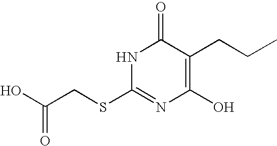

10

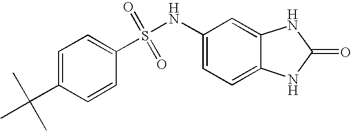

11

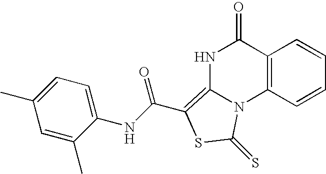

12

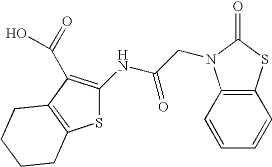

13

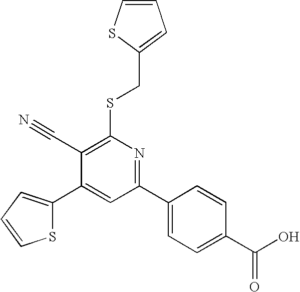

14
Compounds 15-26

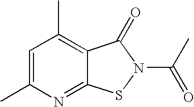

15

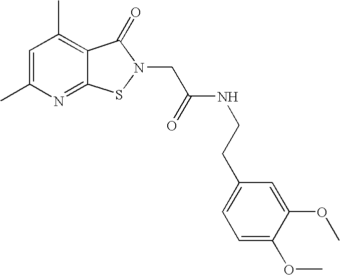

16

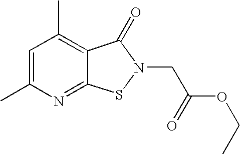

17

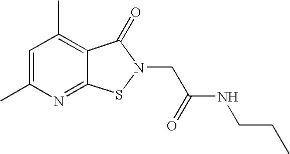

18

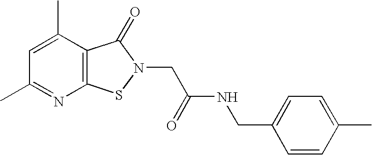

19

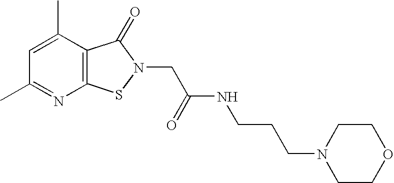

20

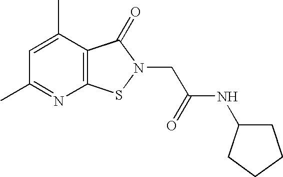

21

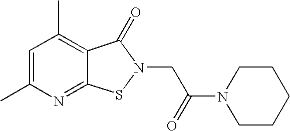

22

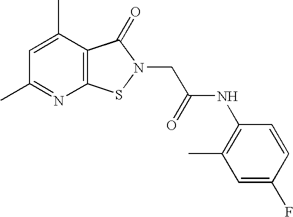

23

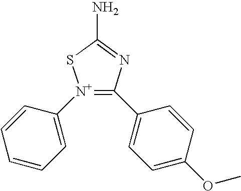

24

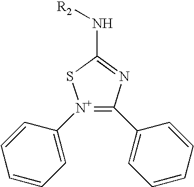

25

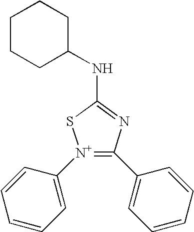

26

Compounds presented herein as compounds 4-26 may be useful to inhibit O-GlcNAcylation of proteins by OGT. Compounds such as those exemplified by compounds 4-14 may be used to treat an OGT-associated disease or disorder in subjects in need of such treatment. It will be understood by those of ordinary skill in the art that analogs, derivatives, or variants of one or more core compounds or other compounds disclosed herein may be used as inhibitors of OGT, and in some aspects of the invention, such inhibitors may be used to treat or prevent an OGT-associated disease or condition, e.g., diabetes, pre-diabetes, etc.

A compound of the invention may be an isolated compound. By “isolated”, it is meant present in sufficient quantity to permit its identification or use according to the procedures described herein. Because an isolated material may be admixed with a carrier in a preparation, such as, for example, for adding to a sample or for analysis, the isolated material may comprise only a small percentage by weight of the preparation.

In some aspects of the invention, one or more of compounds 4-14 may be administered to a subject that is free of indications for a previously determined use of the compounds. By “free of indications for a previously determined use”, it is meant that the subject does not have symptoms that call for treatment with one or more of the compounds of the invention for a previously determined use of that compound, other than the indication that exists as a result of this invention. As used herein the term “previously determined use” of a compound means the use of the compound that was previously identified. Thus, the previously determined use is not the use of inhibiting OGT activity and/or the O-GlcNAcylation of proteins.

The methods of the invention include administration of an OGT-inhibiting compound that preferentially targets neuronal or vascular cells and/or tissues or other specific cell or tissue types. In addition, the compounds can be specifically targeted to neuronal or vascular tissue or other specific tissue types. The targeting may be done using various delivery methods, including, but not limited to: administration to neuronal or vascular tissue or other specific target tissue, the addition of targeting molecules to direct the compounds of the invention to neuronal or other tissues (e.g. glial cells, nerve cells, vascular cells, etc.). Additional methods to specifically target compounds and compositions of the invention to specific tissues, such as neuronal tissues, vascular tissues, or other types of tissues may also be used with the compounds and compositions of the invention, and are known to those of ordinary skill in the art.

The invention involves, in part, compounds that inhibit OGT activity in cells, tissues, and/or subjects an the use of such compounds to inhibit OGT. The OGT inhibitors of the invention may be used for treatment of cells, tissues, and/or subjects and for research purposes. As used herein, the term “OGT activity” means the O-GlcNAcylation of proteins. It is understood that hyper O-GlcNAcylation of proteins, which is the O-GlcNAcylation of proteins at a level above a normal level, may occur in certain diseases, including, but not limited to, diabetes and pre-diabetes conditions. The hyper O-GlcNAcylation of proteins may also occur in other conditions and in other tissues as a result of disease and may result in cell and tissue damage. For example, levels of O-GlcNAcylation that are above normal levels may result in insulin resistance.

The OGT-inhibiting compounds of the invention may be administered to a subject to reduce the risk of a OGT-associated disorder. Reducing the risk of a disorder associated with above-normal OGT activity means using treatments and/or medications to reduce OGT activity levels, therein reducing, for example, the subject's risk of insulin resistance, vascular complications including but not limited to: diabetic nephropathy, diabetic retinopathy, macro-vascular disease, micro-vascular disease, and diabetic neuropathy.

As used herein, the term “subject” means any mammal that may be in need of treatment with an OGT-inhibiting compound of the invention. Subjects include but are not limited to: humans, non-human primates, cats, dogs, sheep, pigs, horses, cows, rodents such as mice, rats, etc.

As used herein the term “inhibit” means to reduce the amount of OGT activity and/or O-GlcNAcylation to a level or amount that is statistically significantly less than an initial level, which may be a control level of OGT activity and/or O-GlcNAcylation. As used herein, an initial level may be a level in a cell, tissue, or subject not contacted with an OGT-inhibiting compound of the invention. In some cases, the decrease in the level of OGT activity and/or O-GlcNAcylation means the level of OGT activity and/or O-GlcNAcylation is reduced from an initial level to a level significantly lower than the initial level. In some cases, the reduced level may be zero.

In some embodiments, a control level of OGT activity and/or O-GlcNAcylation is the level that represents the normal level of OGT activity and/or O-GlcNAcylation in a cell, tissue, and/or subject. For example, a control level may be a level that is not associated with hyper O-GlcNAcylation and cell damage and/or death. In some instances, a control level will be the level in a disorder-free cell, tissue, or subject, that does not have abnormally high levels of OGT activity (e.g. hyper OGT activity) and/or abnormally high levels of O-GlcNAcylation, and may be useful, for example, to monitor a change in the level of OGT activity and/or O-GlcNAcylation in a cell. In other instances a control level of OGT activity and/or O-GlcNAcylation will be the level in a cell, tissue, or subject with a disorder such as pre-diabetes or diabetes, etc. that is associated with OGT activity and/or O-GlcNAcylation, and may be useful, for example, to monitor a decrease in the level of OGT activity and/or O-GlcNAcylation in a cell, tissue, or subject. In other embodiments, a control level of OGT activity and/or O-GlcNAcylation will be the level in a cell, tissue, or subject with a disorder such as a neurodegenerative disorder, e.g. Alzheimer's disease, or cancer, etc. and may be useful, for example, to monitor a change in the level of OGT activity and/or O-GlcNAcylation in a cell, tissue, and/or subject. These, and other, types of control levels are useful in assays to assess the efficacy of an OGT-activity modulating and/or O-GlcNAcylation modulating compound of the invention.

It will be understood by one of ordinary skill in the art that a control level of OGT activity and/or O-GlcNAcylation may be a predetermined value, which can take a variety of forms. It can be a single value, such as a median or mean. It can be established based upon comparative groups, such as in disease-free groups that have normal levels of OGT activity and/or O-GlcNAcylation. Other comparative groups may be groups of subjects with specific disorders, e.g. pre-diabetes, insulin resistance, type 1 diabetes, type 2 diabetes, complications of diabetes, neurodegenerative disorders, Alzheimer's disease, cancer, etc. It will be understood that disease-free cells and/or tissues may be used as comparative groups for cells or tissues that have a OGT activity-related disorder and/or an O-GlcNAcylation-associated disorder.

In some embodiments, of methods and assays to evaluate an effect of a candidate compound on OGT activity, the results in a test sample may be compared to the results in a control sample that is essentially identical to the test sample, but lacks the candidate compound. The displacement of the probe from a test sample versus the displacement of the probe from a control sample may be compared as a measure of the inhibitor effect of the candidate compound on OGT activity. Those of ordinary skill in the art will recognize the manner of using control values and samples in conjunction with assays and methods of the invention.

Similarly, in some embodiments of methods and assays of the invention, a control amount or level of OGT activity may be the amount in an control sample that is substantially identical to a test sample, but the control sample lacks one or more constituents that are included in the test sample. For example, a test sample for an assay to confirm whether a candidate compound is an OGT inhibitor, may include: a candidate inhibitor compound and (i) an OGT acceptor polypeptide (e.g., a polypeptide set forth as SEQ D NO:2); (ii) a UDP-GlcNAc probe comprising detectably labeled GlcNAc, and (iii) OGT; and a control sample may include (i) an OGT acceptor polypeptide set forth as SEQ ID NO:2, (ii) a UDP-GlcNAc probe comprising detectably labeled GlcNAc, and (iii) OGT; but may not include the candidate inhibitor compound. The level of OGT activity between such samples can be compared to each other to determine the effect of the candidate OGT inhibitor compound on the OGT activity to see whether the candidate inhibitor compound reduces the OGT activity.

In some embodiments, a compound that inhibits and thereby reduces the level of OGT activity and/or O-GlcNAcylation is a compound that reduces the likelihood or risk of having an O-GlcNAcylation-associated or OGT-associated disease or disorder. A level of OGT activity and/or O-GlcNAcylation in a cell, tissue, and/or subject may be one that is below the OGT activity level in cells, tissues, and/or subjects with diabetes or pre-diabetes, e.g. may be a level that is clinically asymptomatic, but may still be treated and further reduced by administration of a compound of the invention. The invention relates in part to the administration of an OGT-inhibiting and/or O-GlcNAcylation-inhibiting compound of the invention to a cell, tissue, and/or subject in an amount effective to reduce OGT activity and/or O-GlcNAcylation in cells, tissues, and/or subjects with an OGT-associated and/or O-GlcNAcylation-associated disease or disorder.

In some aspects of the invention, OGT-modulating (inhibiting or enhancing) compounds include functional analogs, derivatives, and/or variants of the OGT-modulating compounds of the invention specifically disclosed herein. Thus, the term “OGT-modulating compounds” may include functional analogs, derivatives, and/or variants of the compounds presented herein as compounds 4-26. For example, functional analogs, derivatives, and variants of the OGT-modulating compounds of Table 1 may be made to enhance a property of a compound, such as stability. Functional analogs, derivatives, and variants of the compounds of Table 1 may also be made to provide a novel activity or property to a compound of Table 1, for example, to enhance detection, to enhance potency, to reduce side effects, etc. In some embodiments of the invention, modifications to an OGT-modulating compound of the invention, can be made to the structure or side groups of the compound and can include one or more deletions, substitutions, and additions of atoms, or side groups. Alternatively, modifications can be made by addition of a linker molecule, addition of a detectable moiety, such as biotin or a fluorophore, chromophore, enzymatic, and/or radioactive label, and the like.

Analogs of the OGT-modulating compounds of Table 1 that retain some or all of the OGT-modulating properties also can be used in accordance with the invention. In some embodiments, an analog of a molecule may have a higher level of OGT-modulating activity than the original compound. Chemical groups that can be added to or substituted in the molecules include: hydrido, alkyl, alkenyl, alkynyl, cycloalkyl, heterocycyl, aryl, heteroaryl, alkoxy, aryloxy, sulfoxy, acyl, amino, acyloxy, acylamino, carboalkoxy, carboxyamido, carboxyamido, halo and thio groups. Substitutions can replace one or more chemical groups or atoms on the molecules provided herein, e.g., compounds 4-26.

Molecular terms, when used in this application, have their common meaning unless otherwise specified. The term “hydrido” denotes a single hydrogen atom (H). The term “acyl” is defined as a carbonyl radical attached to an alkyl, alkenyl, alkynyl, cycloalkyl, heterocyclyl, aryl or heteroaryl group, examples of such radicals being acetyl and benzoyl. The term “amino” denotes a nitrogen radical containing two substituents independently selected from the group consisting of hydrido, alkyl, cycloalkyl, heterocyclyl, aryl, and heteroaryl. The term “acyloxy” denotes an oxygen radical adjacent to an acyl group. The term “acylamino” denotes a nitrogen radical adjacent to an acyl group. The term “carboalkoxy” is defined as a carbonyl radical adjacent to an alkoxy or aryloxy group. The term “carboxyamido” denotes a carbonyl radical adjacent to an amino group. The term “carboxy” embraces a carbonyl radical adjacent to an alkyl, alkenyl, alkynyl, cycloalkyl, heterocyclyl, aryl or heteroaryl group. The term “halo” is defined as a bromo, chloro, fluoro or iodo radical. The term “thio” denotes a radical containing a substituent group independently selected from hydrido, alkyl, cycloalkyl, heterocyclyl, aryl and heteroaryl, attached to a divalent sulfur atom, such as, methylthio and phenylthio.

The term “alkyl” is defined as a linear or branched, saturated radical having one to about ten carbon atoms unless otherwise specified. Preferred alkyl radicals are “lower alkyl” radicals having one to about five carbon atoms. One or more hydrogen atoms of an alkyl can also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of alkyl groups include methyl, tert-butyl, isopropyl, and methoxymethyl.

The term “alkenyl” embraces linear or branched radicals having two to about twenty carbon atoms, preferably three to about ten carbon atoms, and containing at least one carbon-carbon double bond. One or more hydrogen atoms of an alkenyl can also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of alkenyl groups include ethylenyl or phenyl ethylenyl.

The term “alkynyl” denotes linear or branched radicals having from two to about ten carbon atoms, and containing at least one carbon-carbon triple bond. One or more hydrogen atoms of an alkynyl can also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of alkynyl groups include propynyl.

The term “aryl” denotes aromatic radicals in a single or fused carbocyclic ring system, having from five to twelve ring members. One or more hydrogen atoms of an aryl may also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of aryl groups include phenyl, naphthyl, biphenyl, and terphenyl. “Heteroaryl” embraces aromatic radicals which contain one to four hetero atoms selected from oxygen, nitrogen and sulfur in a single or fused heterocyclic ring system, having from five to fifteen ring members. One or more hydrogen atoms of an heteroaryl may also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of heteroaryl groups include, pyridinyl, thiazolyl, thiadiazoyl, isoquinolinyl, pyrazolyl, oxazolyl, oxadiazoyl, triazolyl, and pyrrolyl groups.

The term “cycloalkyl” is defined as a saturated or partially unsaturated carbocyclic ring in a single or fused carbocyclic ring system having from three to twelve ring members. One or more hydrogen atoms of a cycloalkyl may also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of a cycloalkyl group include cyclopropyl, cyclobutyl, cyclohexyl, and cycloheptyl.

The term “heterocyclyl” embraces a saturated or partially unsaturated ring containing zero to four hetero atoms selected from oxygen, nitrogen and sulfur in a single or fused heterocyclic ring system having from three to twelve ring members. One or more hydrogen atoms of a heterocyclyl may also be replaced by a substituent group selected from acyl, amino, acylamino, acyloxy, carboalkoxy, carboxy, carboxyamido, cyano, halo, hydroxy, nitro, thio, alkyl, alkenyl, alkynyl, cycloalkyl, heterocyclyl, aryl, heteroaryl, alkoxy, aryloxy, sulfoxy, and formyl. Examples of a heterocyclyl group include morpholinyl, piperidinyl, and pyrrolidinyl. The term “alkoxy” denotes oxy-containing radicals substituted with an alkyl, cycloalkyl or heterocyclyl group. Examples include methoxy, tert-butoxy, benzyloxy and cyclohexyloxy. The term “aryloxy” denotes oxy-containing radicals substituted with an aryl or heteroaryl group. Examples include phenoxy. The term “sulfoxy” is defined as a hexavalent sulfur radical bound to two or three substituents selected from the group consisting of oxo, alkyl, cycloalkyl, heterocyclyl, aryl and heteroaryl, wherein at least one of said substituents is oxo.

The OGT-modulating compounds of the invention also include, but are not limited to any pharmaceutically acceptable salts, esters, or salts of an ester of each compound. Examples of salts that may be used, which is not intended to be limiting include: chloride, acetate, hydrochloride, methanesulfonate or other salt of a compound of Table 1 or a functional analog, derivative, variant, or fragment of the compound.

Derivatives of the compounds of Table 1 include compounds which, upon administration to a subject in need of such administration, deliver (directly or indirectly) a pharmaceutically active OGT-modulating (e.g. inhibiting) compound as described herein. An example of pharmaceutically active derivatives of the invention includes, but is not limited to, pro-drugs. A pro-drug is a derivative of a compound that contains an additional moiety that is susceptible to removal in vivo yielding the parent molecule as a pharmacologically active agent. An example of a pro-drug is an ester that is cleaved in vivo to yield a compound of interest. Pro-drugs of a variety of compounds, and materials and methods for derivatizing the parent compounds to create the pro-drugs, are known to those of ordinary skill in the art and may be adapted to the present invention.

Analogs, variants, and derivatives of the compounds of the invention set forth in Table 1 may be identified using standard methods known to those of ordinary skill in the art. Useful methods involve identification of compounds having similar chemical structure, similar active groups, chemical family relatedness, and other standard characteristics. For the purposes of this invention, the chemical elements are identified in accordance with the Periodic Table of the Elements, CAS version, Handbook of Chemistry and Physics 75^{th} Ed, inside cover, and specific functional groups are defined as described therein. Additionally, general principles of organic chemistry, as well as specific functional moieties and reactivity, are described in “Organic Chemistry”, Thomas Sorrell, University Science Books, Sausalito. 1999, the contents of which are incorporated herein by reference in their entirety.

Using the structures of the compounds disclosed herein, one of ordinary skill in the art is enabled to make predictions of structural and chemical motifs for analogs, variants, and/or derivatives that possess similar functions of the compounds disclosed in Table 1. Using structural motifs as search, evaluation, or design criteria, one of ordinary skill in the art is enabled to identify classes of compounds (functional derivatives, analogs, and/or variants of the OGT-modulating compounds) that possess the inhibitory function of the compounds disclosed herein. These compounds may be synthesized using standard synthetic methods and tested for activity as described herein. Examples of derivatives, analogs, and variants are known to those of skill in the art.

The invention also involves methods for determining the functional activity of OGT-modulating compounds described herein. The function or status of a compound as a OGT-modulating compound can be determined according to assays known and those described herein. For example, the purified enzyme assays described herein may be used to assess compounds for OGT-modulating ability. In addition, cell assays can be used to assess OGT-modulating ability of compounds. For example, cells can be contacted with a candidate OGT-modulating compound under conditions that produce OGT activity, and standard procedures can be used to determine whether OGT activity is modulated (e.g. inhibited) by the compound and/or whether O-GlcNAcylation is modulated by the candidate compound. Such methods may also be utilized to determine the status of analogs, variants, and derivatives as inhibitors of OGT activity and O-GlcNAcylation. Although not intended to be limiting, examples of methods with which the ability of an OGT-modulating compound to modulate or change OGT activity and/or O-GlcNAcylation can be tested, are purified enzyme assays, in vitro and in vivo assay systems provided herein in the Examples section.

Using such assays the level of OGT activity (e.g. binding and/or catalytic activity) and/or O-GlcNAcylation can be measured in a system both before and after contacting the system with a candidate OGT-modulating compound as an indication of the effect of the compound on the level of OGT activity and/or O-GlcNAcylation. Secondary screens may further be used to verify the efficacy of compounds identified as modulators of OGT activity and/or O-GlcNAcylation. Examples of initial displacement screens and secondary screens are provided in the Examples section. It will be understood by those of ordinary skill in the art that OGT molecules and OGT substrates that may be used in methods and assays of the invention include any suitable OGT molecules and OGT substrates and that the ones specifically disclosed herein are exemplary OGT molecules and OGT substrates and are not intended to be limiting.

In addition, derivatives, analogs, and variants of OGT-modulating compounds can be tested for their OGT activity modulation and/or modulation of O-GlcNAcylation by using an activity assay (see examples). An example of an assay method, although not intended to be limiting, are purified enzyme assays, such as the displacement assays provided herein. In displacement assays, OGT and a probe that binds to OGT are mixed and a derivative, analog, or variant of an OGT-inhibiting compound is added. Displacement of the probe from the OGT is then detected as a measure of the ability of the candidate compound to inhibit OGT. Other cells and tissue-based assays may include contacting a tissue or cell sample with an OGT-modulating compound and determining the compound's modulatory activity as described herein. Contacting a similar cell or tissue sample with an analog of the OGT-modulating compound, determining its activity, and then comparing the two activity results can serve as a measure of the efficacy of the derivative, variant, and/or analog's OGT-modulating activity.

In addition to the in vitro assays described above and the purified enzyme assays and other assays described in the Examples section, an in vivo assay may be used to determine the functional activity of OGT-modulating compounds described herein. In such assays, animal models of OGT-associated disease and/or O-GlcNAcylation-associated disease can be treated with an OGT-modulating compound of the invention. OGT-modulation (e.g. inhibition) may be assayed using methods described herein, which may include labeling or imaging methods. Additionally, animals with and without OGT-modulating compound treatment can be examined for behavior and/or survival as an indication of the effectiveness and/or efficacy of the compound. Behavior may be assessed by examination of symptoms of abnormal OGT activity and/or O-GlcNAcylation as described herein. These measurements can then be compared to corresponding measurements in control animals. For example, test and control animals may be examined following administration of an OGT-modulating compound of the invention. In some embodiments, test animals are administered an OGT-modulating compound of the invention and control animals are not. Any resulting change in OGT activity and/or O-GlcNAcylation can then be determined for each type of animal using known methods in the art as described herein. Such assays may be used to compare levels of OGT activity and/or O-GlcNAcylation activity in animals administered the candidate OGT-modulating compound to control levels of OGT activity and/or O-GlcNAcylation in animals not administered the OGT-modulating compound as an indication that the putative OGT-modulating compound is effective to alter (e.g. increase or decrease) OGT activity and/or O-GlcNAcylation. In other embodiments, a candidate OGT-modulating compound may be administered to both a test and control animal and the results on OGT activity and/or O-GlcNAcylation may be compared as a measure of the efficacy of the compound.

Once one or more OGT-modulating compounds are verified as inhibiting OGT activity and/or O-GlcNAcylation using art-known assays or assays as described herein (e.g., in Examples), further biochemical and molecular techniques may be used to identify the targets of these compounds and to elucidate the specific roles that these target molecules play in the process of OGT activity and/or O-GlcNAcylation in associated diseases and/or disorders. An example, though not intended to be limiting, is that the compound(s) may be labeled and contacted with a cell to identify the host cell proteins with which these compounds interact. Such proteins may be purified, e.g., by labeling the compound with an immunoaffinity tag and applying the protein-bound compound to an immunoaffinity column.

An OGT-modulating compound of the invention (e.g., an OGT inhibitor) may be used to treat a subject with an OGT-associated disease or disorder. As used herein, the term “treat” includes active treatment of a subject that has an OGT disease or disorder (e.g., a subject diagnosed with such a condition) and also includes prophylactic treatment of a subject who is has not yet been diagnosed. Compounds of the invention may administered prophylactically to a subject at risk of an OGT-associated disorder or disorder. Determination of a subject at risk for an OGT-associated disease or disorder, and/or the determination of a diagnosis of an OGT-associated disease or disorder in a subject, may be done by one of ordinary skill in the art using routine methods.

An OGT-modulating compound of the invention may be delivered to a cell using standard methods known to those of ordinary skill in the art. Various techniques may be employed for introducing OGT-modulating compounds of the invention to cells, depending on whether the compounds are introduced in vitro or in vivo in a host.

When administered, the OGT-modulating compounds (also referred to herein as therapeutic compounds and/or pharmaceutical compounds) of the present invention are administered in pharmaceutically acceptable preparations. Such preparations may routinely contain pharmaceutically acceptable concentrations of salt, buffering agents, preservatives, compatible carriers, and optionally other therapeutic agents.

The term “pharmaceutically acceptable” carrier means a non-toxic material that does not interfere with the effectiveness of the biological activity of the active ingredients. The characteristics of the carrier will depend on the route of administration.

The therapeutics of the invention can be administered by any conventional route, including injection or by gradual infusion over time. The administration may for example, be oral, intravenous, intraperitoneal, intrathecal, intramuscular, intranasal, intracavity, subcutaneous, intradermal, mucosal, transdermal, or transdermal.

The therapeutic compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

In some embodiments of the invention, an OGT-modulating compound of the invention may be delivered in the form of a delivery complex. The delivery complex may deliver the OGT-modulating compound into any cell type, or may be associated with a molecule for targeting a specific cell type. Examples of delivery complexes include an OGT-modulating compound of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell specific receptor). Some complexes may be sufficiently stable in vivo to prevent significant uncoupling prior to internalization by the target cell. However, the complex can be cleavable under appropriate conditions within the cell so that the OGT-modulating compound is released in a functional form.

An example of a targeting method, although not intended to be limiting, is the use of liposomes to deliver an OGT-modulating compound of the invention into a cell. Liposomes may be targeted to a particular tissue, such neuronal cells, (e.g. hippocampal cells, etc), or other cell type, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

For certain uses, it may be desirable to target the compound to particular cells, for example specific neuronal cells, including specific tissue cell types, e.g. tissue-specific nervous system cells. In some embodiments, it may be desirable to target an OGT-modulating compound to another cell type, including, but not limited to, cardiac cells, pancreatic cells, vascular cells, etc. In such instances, a vehicle (e.g. a liposome) used for delivering an OGT-modulating compound of the invention to a cell type (e.g. a neuronal cell, vascular cell, etc.) may have a targeting molecule attached thereto that is an antibody specific for a surface membrane polypeptide of the cell type or may have attached thereto a ligand for a receptor on the cell type. Such a targeting molecule can be bound to or incorporated within the OGT-modulating compound delivery vehicle. Where liposomes are employed to deliver an OGT-modulating compound of the invention, proteins that bind to a surface membrane protein associated with endocytosis may be incorporated into the liposome formulation for targeting and/or to facilitate uptake.

Liposomes are commercially available from Invitrogen, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include, but are not limited to, polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. WO 95/24929, entitled “Polymeric Gene Delivery System”. describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in WO 95/24929. The polymeric matrix preferably is in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used. The polymeric matrix composition can be selected to have both favorable degradation rates and also to be formed of a material which is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents and compounds of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents and/or compounds of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of subjects with an established neurological disorder or complication of diabetes as well as subjects at risk of developing a neurological disorder, insulin resistance, pre-diabetes, diabetes, or a complication of diabetes.

“Long-term” release, as used herein, means that the implant is constructed and arranged to deliver therapeutic levels of the active ingredient for at least 7 days, and preferably 30-60 days, and most preferably months or years. The implant may be positioned at or near the site of the neurological damage or the area of the brain or nervous system affected by or involved in the neurodegenerative disorder. Long-term release implants may also be used in non-neuronal tissues and organs to allow regional administration of an OGT-modulating compound of the invention. Long-term sustained release implants are well known to those of ordinary skill in the art and include some of the release systems described above.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, stimulates the desired response. In the case of treating a disorder or condition that is associated with abnormal OGT activity and/or abnormal O-GlcNAcylation, desired response is reducing the onset, stage or progression of the abnormal OGT activity and/or O-GlcNAcylation and associated effects. This may involve only slowing the progression of the damage temporarily, although more preferably, it involves halting the progression of the damage permanently. An effective amount for treating abnormal OGT activity and/or O-GlcNAcylation is that amount that alters (increases or reduces) the amount or level of OGT activity and/or O-GlcNAcylation, when the cell or subject is a cell or subject with an OGT-associated disease or disorder, with respect to that amount that would occur in the absence of the active compound.

The invention involves, in part, the administration of an effective amount of an OGT-modulating compound of the invention. The OGT-modulating compounds of the invention are administered in effective amounts. Typically effective amounts of an OGT-modulating compound will be determined in clinical trials, establishing an effective dose for a test population versus a control population in a blind study. In some embodiments, an effective amount will be that amount that diminishes or eliminates an OGT-associated and/or a O-GlcNAcylation-associated disease or disorder and its effects in a cell, tissue, and/or subject. Thus, an effective amount may be the amount that when administered reduces the amount of cell and or tissue damage and/or cell death from the amount that would occur in the subject or tissue without the administration of a OGT-modulating compound of the invention.

The pharmaceutical compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days. It will be recognized by those of skill in the art that some of the OGT-modulating compounds may have detrimental effects at high amounts. Thus, an effective amount for use in the methods of the invention may be optimized such that the amount administered results in minimal negative side effects and maximum OGT modulation.

The absolute amount will depend upon a variety of factors, including the material selected for administration, whether the administration is in single or multiple doses, and individual subject parameters including age, physical condition, size, weight, and the stage of the disease or disorder. These factors are well known to those of ordinary skill in the art and can be addressed with no more than routine experimentation.

The pharmaceutical compounds of the invention may be administered alone, in combination with each other, and/or in combination with other drug therapies that are administered to subjects with OGT-activity-associated diseases. Additional drug therapies (for active treatment and/or prophylaxis) that may be administered with pharmaceutical compounds of the invention include, art-known methods of OGT-associated disorders such as treatments for diabetes, complications of diabetes, insulin resistance, neurodegenerative disease, cancer, etc. Alternative drug therapies are known to those of ordinary skill in the art and are administered by modes known to those of skill in the art. The drug therapies are administered in amounts that are effective to achieve the physiological goals (to reduce symptoms and damage from OGT-associated disease or disorder in a subject, e.g. cell damage and/or cell death), in combination with the pharmaceutical compounds of the invention. Thus, it is contemplated that the alternative drug therapies may be administered in amounts which are not capable of preventing or reducing the physiological consequences of the OGT-associated disease and/or disorder when the drug therapies are administered alone, but which are capable of preventing or reducing the physiological consequences of an OGT-associated disease and/or disorder when administered in combination with one or more OGT-modulating compounds of the invention Examples of alternative drug therapies for regulating blood sugar levels, e.g. therapies for pre-diabetes and/or diabetes, include oral therapies with hypoglycemic agents an/or oral anti-diabetic agents, injectable therapies, and the like. Non-drug therapies for regulating blood sugar level include, but are not limited to, diatetic and/or exercise control measures.

Diet and exercise alterations include, but are not limited to, reducing caloric intake, and/or increasing fiber intake, and/or decreasing fat intake, and/or increasing exercise level.

Oral drug therapies for regulating blood sugar levels include hypoglycemic agents that may include, but are not limited to: Acarbose; Acetohexamide; Chlorpropamide; Darglitazone Sodium: Glimepiride; Glipizide; Glyburide, Repaglinide; Troglitazone; Tolazamide; Tolbutamide.

Oral drug therapies for regulating blood sugar levels include antidiabetic agents that may include but are not limited to: Acarbose, Acetohexamide; Buformin; Butoxamine Hydrochloride; Camiglibose; Chlorpropamide; Ciglitazone; Englitazone Sodium; Etoformin Hydrochloride; Gliamilide; Glibomuride; Glicetanile Gliclazide Sodium; Gliflumide; Glipizide; Glucagon; Glyburide; Glyhexamide; Glymidine Sodium; Glyoctamide; Glyparamide; Insulin; Insulin, Dalanated; Insulin Human; Insulin Human, Isophane; Insulin Human Zinc; Insulin Human Zinc, Extended; Insulin, Isophane; Insulin Lispro; Insulin, Neutral; Insulin Zinc; Insulin Zinc, Extended; Insulin Zinc, Prompt; Linogliride; Linogliride Fumarate; Metformin; Methyl Palmoxirate; Palmoxirate Sodium; Pioglitazone Hydrochloride; Pirogliride Tartrate; Proinsulin Human; Repaglinide; Seglitide Acetate, Tolazamide; Tolbutamide; Tolpyrramide; Troglitazone; Zopolrestat.

Injectable therapies for regulating blood sugar levels may include, but are not limited to:

### Fast-Acting Insulin:

Insulin Injection: regular insulin; Prompt Insulin Zinc Suspension; Semilente® insulin. These categories include preparations such as: Humalog® Injection; Humulin® R; Iletin II; Novolin R. Purified Pork Regular Insulin; Velosulin BR Human Insulin;

### Intermediate-Acting Insulin:

Isophane Insulin Suspension: NPH insulin, isophane insulin; Insulin Zinc Suspension Lente® Insulin. These categories include preparations such as: Humulin® L; Humulin®R; Humulin® N NPH; Iletin® II, Lente®; Iletin® II, NPH; Novolin® L, Novolin® N, Purified Pork Lente® insulin, Purified Pork NPH isophane insulin;

### Intermediate and Rapid-Acting Insulin Combinations:

Human Insulin Isophane Suspension/Human Insulin Injection. This category includes preparations such as: Humulin® 50/50; Humulin® 70/30; Novolin® 70/30

### Long-Acting Insulin:

Protamine Zinc Insulin Suspension; Extended Insulin Zinc Suspension. These categories include preparations such as: Ultralente® Insulin, Humulin® U.

Diagnostic tests known to those of ordinary skill in the art may be used to assess the level of OGT activity and/or O-GlcNAcylation in a subject and the effects thereof, and to evaluate a therapeutically effective amount of a pharmaceutical compound administered. Examples of diagnostic tests are set forth below. A first determination of OGT activity and/or the effects thereof in a cell and/or tissue may be obtained using one of the methods described herein (or other methods known in the art), and a second, subsequent determination of the level of OGT activity. A comparison of the OGT activity and/or O-GlcNAcylation and/or the effects thereof on the subject at the different time points may be used to assess the effectiveness of administration of a pharmaceutical compound of the invention as a prophylactic or an active treatment of the OGT-associated disease or disorder. Family history or prior occurrence of an OGT-associated disease or disorder, even if the OGT-associated disease or disorder is absent in a subject at present, may be an indication for prophylactic intervention by administering a pharmaceutical compound described herein to reduce or prevent abnormal OGT activity and/or abnormal O-GlcNAcylation.

An example of a method of diagnosis of abnormal OGT activity and/or abnormal O-GlcNAcylation that can be performed using standard methods such as, but not limited to: imaging methods, electrophysiological methods, blood tests, and histological methods. Additional methods of diagnosis and assessment of OGT-associated disease or disorders and the resulting cell death or damage are known to those of skill in the art.

In addition to the diagnostic tests described above, clinical features of OGT-associated diseases and/or disorders can be monitored for assessment of OGT activity following onset of an OGT-associated disease or disorder. These features include, but are not limited to: assessment of the presence of cell damage, cell death, neuronal cell lesions, brain lesions, organ lesions, vascular damage, blood abnormalities, sugar processing abnormalities, and behavioral abnormalities. Such assessment can be done with methods known to one of ordinary skill in the art, such as behavioral testing, blood testing, and imaging studies, such as radiologic studies, CT scans, PET scans, etc.

The invention also provides a pharmaceutical kit comprising one or more containers comprising one or more of the OGT-modulating compounds of the invention and/or formulations of the invention. The kit may also include instructions for the use of the one or more OGT-modulating compounds or formulations of the invention for the treatment of an OGT-associated disease or disorder. The kits of the invention may also comprise one or more containers containing additional drugs for treating an OGT-associated disease or disorder. The invention also includes in some aspects, kits for testing candidate compounds to assess their ability to inhibit OGT activity.

The invention further provides efficient methods of identifying pharmacological compounds or lead compounds and compounds that inhibit (or enhance) OGT activity and/or O-GlcNAcylation. Generally, the screening methods involve assaying for compounds which modulate (enhance or inhibit) the level of OGT activity and/or O-GlcNAcylation. As will be understood by those of ordinary skill in the art, the screening methods may measure the level of OGT activity directly, (e.g. binding and/or catalytic activity). Examples of screening methods are provided in the Examples section. In addition, screening methods may be utilized that measure a secondary effect of OGT activity and/or O-GlcNAcylation, for example, the level of cell damage and/or cell death in a cell or tissue sample or by measuring physiological and/or behavioral characteristics of an OGT-associated disease.

A wide variety of assays for OGT-inhibiting compounds can be used in accordance with this aspect of the invention, including, OGT activity assays, OGT displacement assays, purified enzyme assays, cell-free assays, cell-based assays, cell-viability assays, etc. An example of such an assay that is useful to test candidate OGT-inhibiting compounds is a purified enzyme assay provided in the Examples section. In assays for OGT activity modulating compounds, the assay mixture comprises a candidate compound. Typically, a plurality of assay mixtures is run in parallel with different compound concentrations to obtain a different response to the various concentrations. Typically, one of these concentrations serves as a negative control, i.e., at zero concentration of compound or at a concentration of agent below the limits of assay detection.

It is contemplated that cell-based assays can also be performed to assess the ability of compounds of the invention to inhibit OGT activity. Such cell-based assays can be performed using cell samples and/or cultured cells. Biopsy cells and tissues as well as cell lines grown in culture are useful in the methods of the invention.

Candidate compounds useful in accordance with the invention encompass numerous chemical classes, although typically they are organic compounds. Preferably, the candidate compounds are small organic compounds, i.e., those having a molecular weight of more than 50 yet less than about 2500, preferably less than about 1000 and, more preferably, less than about 500. Candidate compounds comprise functional chemical groups necessary for structural interactions with proteins and/or nucleic acid molecules. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid molecule, the agent typically is a DNA or RNA molecule, although modified nucleic acid molecules are also contemplated.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological compounds may be subjected to directed or random chemical modifications such as acylation, alkylation, esterification, amidification, etc. to produce structural analogs of the compounds. Candidate compounds also include analogs, derivatives, and/or variants of the OGT-modulating compounds described herein.

A variety of other reagents also can be included in the assay mixture. These include reagents such as salts, buffers, neutral proteins (e.g., albumin), detergents, etc. which may be used to facilitate optimal binding, or to reduce non-specific or background interactions of the reaction components. Other reagents that improve the efficiency of the assay such as protease inhibitors, nuclease inhibitors, antimicrobial agents, and the like may also be used.

An exemplary purified enzyme assay that is a displacement OGT-inhibition assay is described in the Examples section herein. An OGT-inhibition assay may be used to identify candidate compounds that inhibit OGT activity and/or O-GlcNAcylation and physiological effects thereof. In general, the mixture of the foregoing assay materials is incubated under conditions whereby, but for the presence of the candidate compound, the probe compound binds to OGT. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the level of probe binding to OGT may be detected by any convenient method available to the user. In some embodiments, a fluorescent detection method may be used for detection. Detection may be effected in any convenient way for the assay. In some embodiments, one of the assay components may comprise, or be coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horseradish peroxidase, etc.).

A variety of methods may be used to detect the label, depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

The invention also includes in part, OGT nucleic acid sequences and polypeptide sequences they encode that are useful in the assays of the invention. One exemplary nucleic acid sequence that is useful in the invention assays is presented herein as SEQ ID NO:1.

The invention, in some aspects, includes the nucleic acids that encode an OGT polypeptide and homologs and alleles of the sequences. In general, homologs and alleles typically will share at least 80%, 85%, 90%, 91%, 92%, 93%, 94%, 95%, 96%, 97%, 98%, 99% nucleotide identity and/or at least 95% amino acid identity to the sequences of an OGT nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library.

The invention also includes degenerate nucleic acids that include alternative codons to those present in the native materials and materials of the invention. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating OGT polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as enzymatic activity, compound binding activity, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the arts Any of the foregoing nucleic acids or polypeptides can be tested by routine experimentation for retention of activity or structural relation to the nucleic acids and/or polypeptides disclosed herein. As used herein the terms: “deletion”, “addition”, and “substitution” mean deletion, addition, and substitution changes to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acids of a sequence of the invention.

According to yet another aspect of the invention, an expression vector comprising any of the isolated nucleic acid molecules of the invention, preferably operably linked to a promoter, is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., -galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

The invention will be more fully understood by reference to the following examples. These examples, however, are merely intended to illustrate the embodiments of the invention and are not to be construed to limit the scope of the invention.

### EXAMPLES

### Example 1

### Introduction

Many nuclear and cytosolic proteins are transiently glycosylated by O-GlcNAc transferase (OGT), which transfers N-acetylglucosamine from UDP-GlcNAc to selected serine and threonine residues. O-GlcNAcylation affects such diverse cellular processes as transcription, translation, organelle targeting, and protein-protein interactions (Zachara, N. E. et al., Chem. Rev. 102: 431-438, 2002.), and is believed to play a role in a variety of signaling cascades that mediate glucose homeostasis and stress responses (Zachara, N. E. et al., Biochim. Biophys. Acta. 1673: 13-28, 2004.). Specific inhibitors of OGT could be valuable tools to probe the biological functions of O-GlcNAcylation, but the inability to obtain significant quantities of enzyme, combined with the lack of a high-throughput assay, has impeded efforts to identify such compounds (Konrad, R. J. et al., Biochem. Biophys. Res. Commun. 293: 207-212, 2002.). Conditions have been discovered for expressing large quantities of the catalytic domain of active OGT; a high-throughput donor displacement assay for the enzyme has been designed; and a set of small-molecule inhibitors of OGT activity have been identified. This work lays a foundation for both structural and functional analysis of the catalytic domain of OGT.

An OGT assay using methods described herein, is a displacement assay in which potential modulatory compounds (e.g., inhibitor compounds) such as small molecules or other molecules are contacted with OGT and an OGT probe under conditions in which OGT and the probe bind. The inhibition of, or interference with, binding (binding displacement) of the probe and the OGT can then be determined as a measure of action of the compound as an OGT inhibitor.

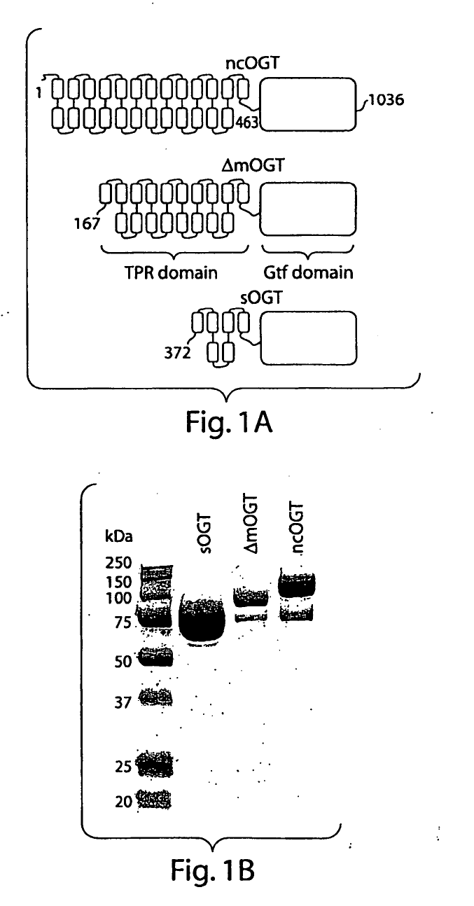

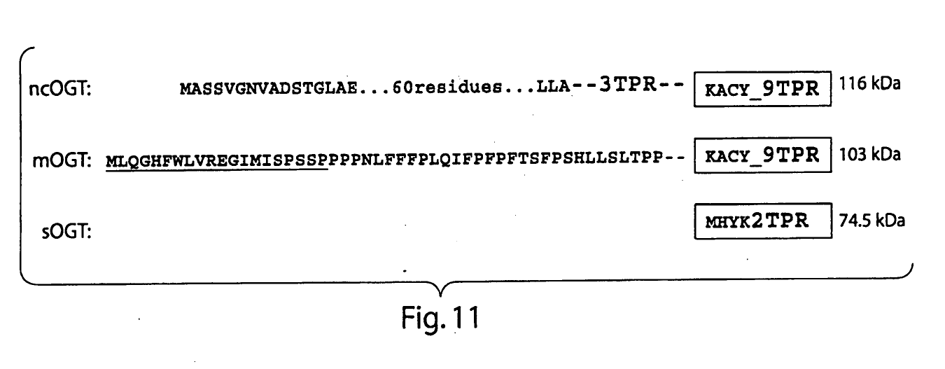

Human, rat, and mouse i genes have previously been expressed in baculovirus (Kreppel, L. K. et al., J. Biol. Chem. 274: 32015-32022, 1999); several mammalian cell lines (Marshall, S. et al., Anal. Biochem. 319: 304-313, 2003; Yang, X. et al., Proc. Natl. Acad. Sci. USA 98:6611-6616, 2001); and Escherichia coli (Lubas, W. A. et al., J. Biol. Chem. 275: 10983-10988, 2000), but good expression levels were not achieved. Therefore, conditions were developed to produce large amounts of pure, active OGT. Production was focused on expression in E. coli because the potential to obtain large amounts of protein is greater than in eukaryotic expression systems. OGT is a bipartite protein consisting of a C-terminal glycosyltransferase (Gtf) domain and an N-terminal protein-protein interaction domain comprised of 12 tetratricopeptide (TPR) repeats. To optimize expression, the gene for human OGT was synthesized using preferred E. coli codons, and then constructs were made based on three known splice variants of OGT [ncOGT (amino acid sequence set forth as Genbank Accession No. O15294, SEQ ID NO:4), mOGT (nucleic acid sequence set forth as SEQ ID NO:1), and sOGT (nucleic acid sequence set forth as SEQ ID NO:3); FIG. 1] (Hanover, J. A. et al., Arch. Biochem. Biophys. 409: 287-297, 2003.). (See also FIG. 11). These constructs were cloned into a modified pET-24b (Novagen) vector for expression as C-terminal His8 fusions. Good expression for all constructs could be achieved by late log induction in BL21-(DE3) at low temperature (16° C.; OD) 1.2; 0.4 mM IPTG), but soluble sOGT was expressed at much higher levels than the other proteins. From 10-12 mg of sOGT/L of culture in >95% purity was obtained after a single step Ni²⁺-IDA IMAC purification. Activity was evaluated using a known peptide substrate. The Kₘ of UDPGlcNAc was found to be 6.7±0.5 μM, nearly identical to the value reported for a construct of the rat enzyme containing six TPRs (Kreppel, L. K. et al., J. Biol. Chem. 274: 32015-32022, 1999.). The specific activity, however, was over 150-fold higher than that reported for the rat enzyme (161 nmol min⁻¹ mg⁻¹ vs 1.06 nmol min⁻¹ mg⁻¹), which may reflect differences in phosphorylation or glycosylation states between OGT produced in E. coli and insect cells (Kreppel, L. K. et al., J. Biol. Chem. 274: 32015-32022, 1999; Tai, H. C. et al., J. Am. Chem. Soc. 126:10500-10501, 2004.). Nup62, a known ncOGT substrate, having an amino acid sequence set forth herein as SEQ D NO:5 (Genbank Accession No. P37198), is also glycosylated by sOGT, and was used as a substrate in some examples herein. This was the first report of sOGT activity in vitro.

### Methods

### Cloning and Expression of OGT

### Gene Synthesis

The gene for human OGT was synthesized using preferred E. coli codons, and constructs were made based on three known splice variants of OGT (ncOGT, mOGT, and sOGT; FIGS. 1 and 11). The amino acid sequence of OGT was back translated using optimal codons for E. coli. Silent mutations that did not introduce poor codons were made to maximize the number of unique restriction sites in the gene. Complete coverage of both strands was achieved with ˜160 40 nucleotide oligomers.

The two strands were staggered by 20 nucleotides, so that each oligomer completely annealed with the two adjacent oligomers of the opposite strand. The oligomers were mixed and the gene was constructed by assembly PCR using Pfu polymerase. The protocol used equimolar concentrations of oligomers (1-5 μM) and consisted of 30 cycles with increasing amplification times (2 seconds to 30 seconds). Using this assembled mixture as a template, PCR with flanking primers amplified the full-length gene. The PCR product was gel-purified, digested with BamH and XhoI and ligated into pBJGI. This plasmid was constructed by amplification of the multi-cloning site of pET36b with S•tag and T7 terminator primers (Novagen), digestion with BlpI and BamHI, followed by ligation with BlpI/BamHI digested pET24b. OGT clones were sequenced and errors (average of 1/kb) were repaired using the Quikchange® (Stratagene) site-directed mutagenesis kit. The nucleic acid sequence of mOGT is set forth as SEQ ID NO:1.

### Expression and Purification of OGT

Plasmids were transformed in BL21 (DE3) cells and grown at 37° C. to an O.D.=1.1-1.3. The culture was cooled to 16° C. and induced with 0.2 mM IPTG. After 24 hours, the cells were pelleted, and frozen at −80° C. Pellets were lysed with Bugbuster® (50 mL/L culture) supplemented with rLysozmeg®, Benzonase® (Novagen), and 40 mM imidazole, pH=7.7. The lysate was clarified by centrifugation at 40,000×g, then loaded onto Ni²⁺-charged IDA column. The column was washed with three column volumes of 50 mM imidazole, pH=7.5, 250 mM NaCl, and the protein was eluted with 250 mM imidazole, pH=7.5, 250 mM NaCl. The elute fraction was dialyzed against 20 mM phosphate, pH=7.5, 150 mM NaCl, and 1 mM EDTA, then supplemented with 500 μM tris(hydroxypropyl)phosphine and stored at −80° C. The dialyzed elute fractions are shown in FIG. 1B.

Radiometric Assay and Kinetics of Purified sOGT and ncOGT

The peptide assay was performed as described herein (see procedure for the Secondary assay below), and the Kₘ of UDP-GlcNAc and K_{cat} were determined by varying the UDP-GlcNAc concentration from 0.5 μM to 60 μM while holding the peptide (SEQ ID NO:2) constant. Nup62 was expressed and purified as previously described by Hanover and co-workers (Lubas, W. A.; Smith, M.; Starr, C. M.; Hanover, J. A.; Biochemistry, 1995, 34, (5), 1686-94) and was stored in 2 M urea at −80° C. The OGT assay with nup62 was carried out as described for the peptide except that the reaction mixture had a final concentration of 500 mM urea. The nup62 reactions were run for 15-30 minutes, spotted on Whatman MM filter paper, washed with 3×10% TCA for 5 minutes, washed with acetone for 5 minutes, and then counted by liquid scintillation counting.

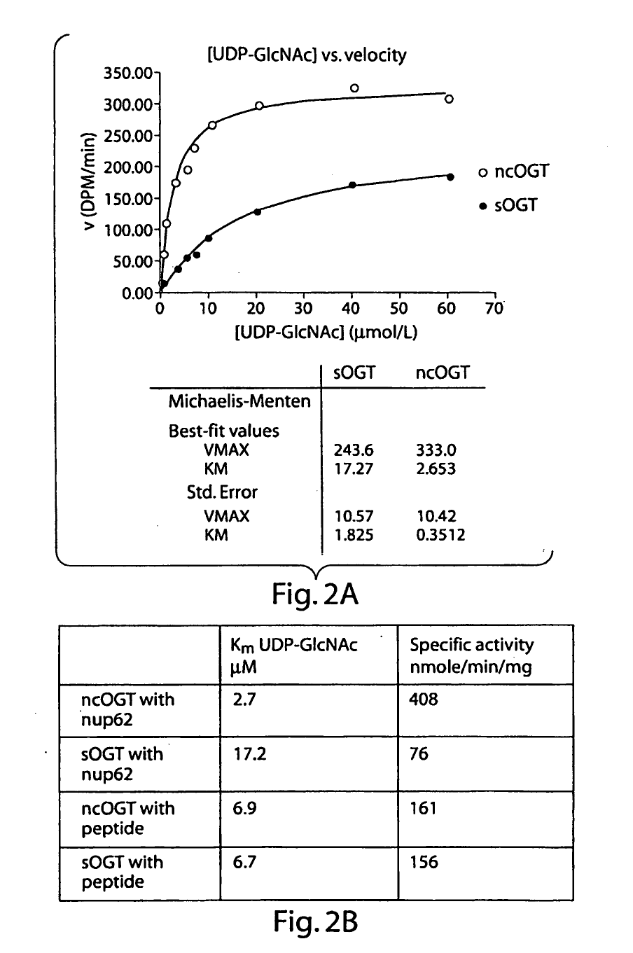

The non-linear regression analysis of UDP-GlcNAc with ncOGT and sOGT and 31 μM nup62 is shown in FIG. 2A. The Kₘ values and the specific activity of both splice variants with peptide and protein substrate are shown in FIG. 2B. Non-linear regression [UDP-GlcNAc] vs. velocity analysis was computed using Prism 4 software (Graphpad, San Diego, Calif.).

### IC₅₀ Determination and Inhibition Kinetics

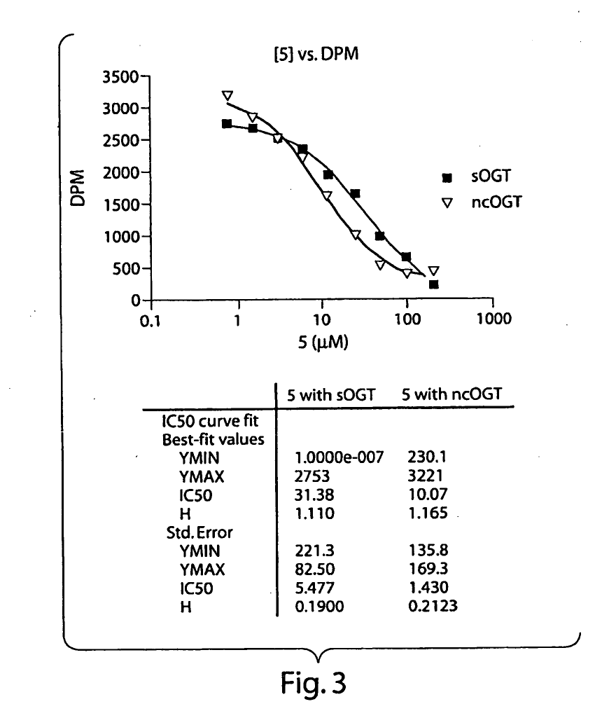

The assay procedure for IC₅₀ measurements is identical to the Secondary Screen Protocol described herein, with the exceptions that the inhibitor concentration was varied from 200 μM to 0.7 μM and either ncOGT or sOGT was used in the reaction. The IC₅₀ curve was fit to the following equation using Prism 4 software (Graphpad) (see FIG. 3).

Y=Ymin+(Ymax−Ymin)/(1+10̂((log(X)−log(IC50))*h))

where: X is the inhibitor concentration, Y is the reaction rate, and h is the hill slope.

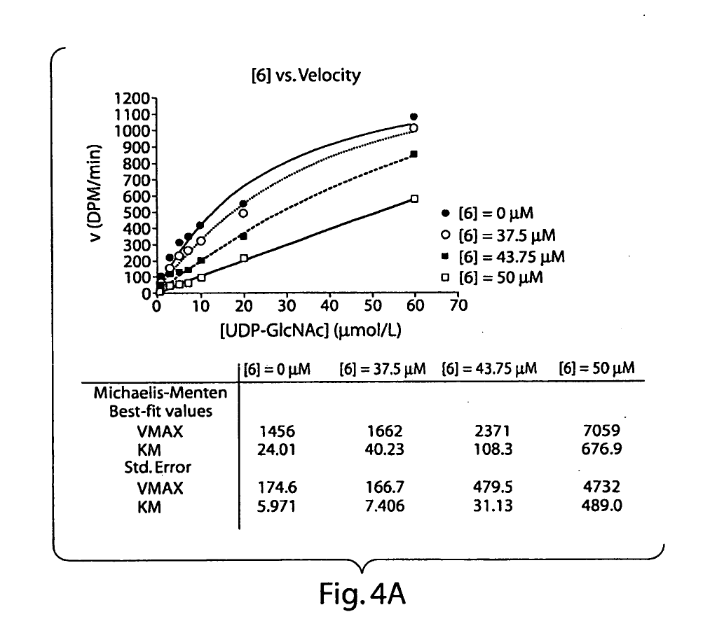

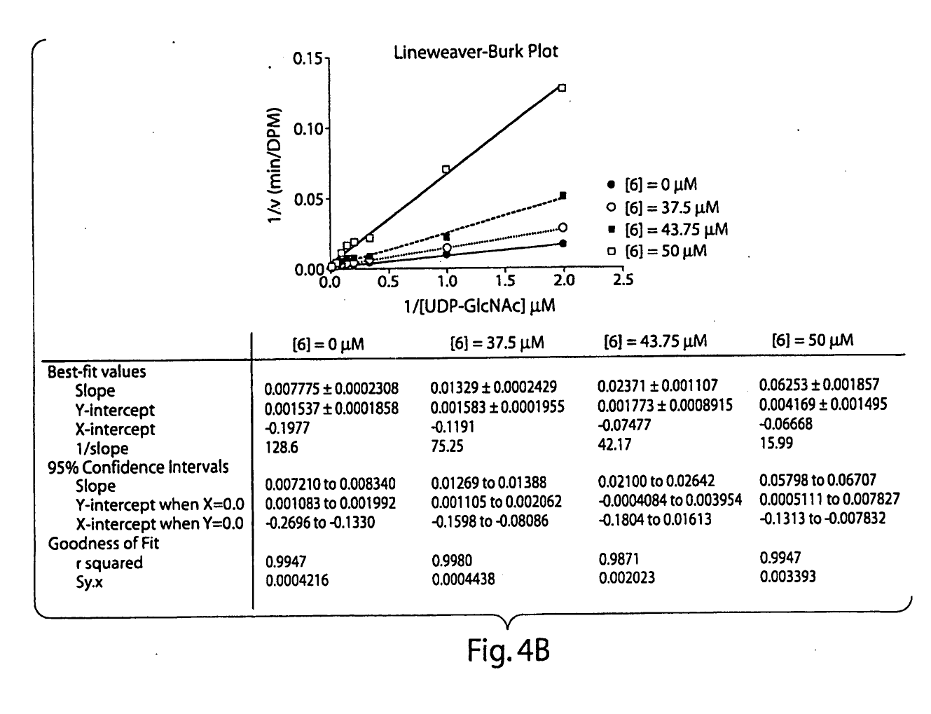

Mode of inhibition studies were carried out using the assay as described above, but the concentration of UDP-¹⁴C-GlcNAc vas varied between 0.5 μM and 60 μM while inhibitor concentration was held constant. Non-linear regression analysis and Lineweaver-Burk plots were computed using Prism 4 software (Graphpad) (See FIG. 4).

### Synthesis of Probes and Assay of OGT Modulating Compounds

### Synthesis of UDP-GlcNAc Analogs (Probes)

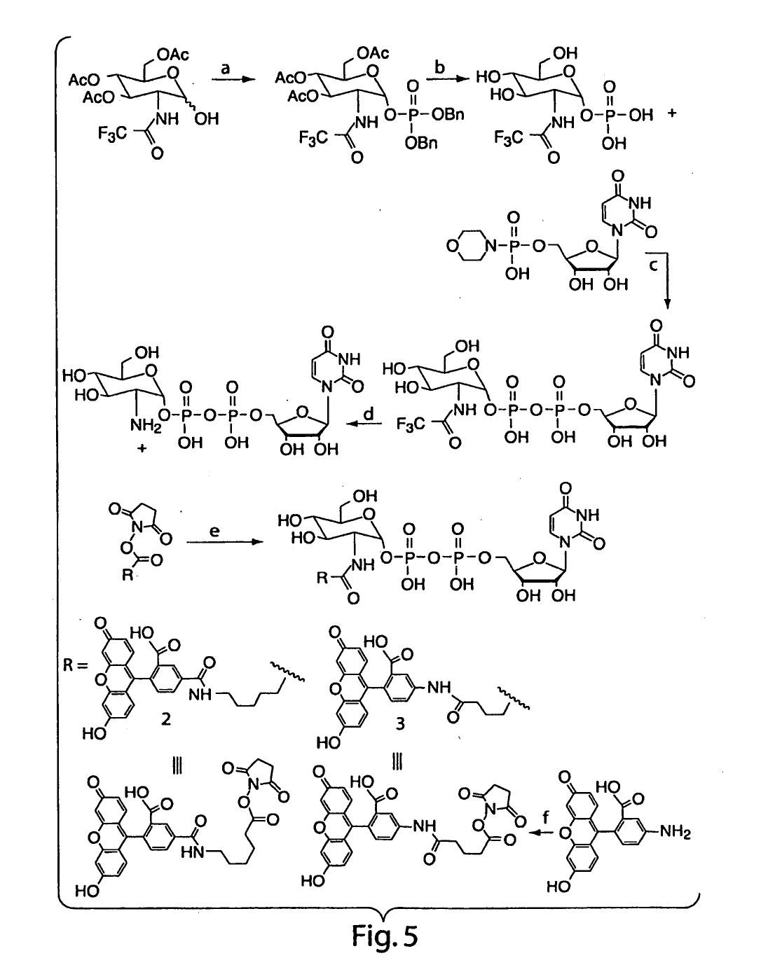

The UDP-GlcNAc probes were synthesized as illustrated in FIG. 5. The reagents and conditions, as illustrated in FIG. 5, were: (a) tetrazole (2 eq.), and dibenzyl N,N-diisopropylphosphoramidite (1.1 eq.), CH₂Cl₂-50° C. to −10° C., 1 hr. followed by MCPBA (2.5 eq.)-60° C. to room temperature, 2.5 hrs., 65% yield. (b) 10% Pd/carbon, MeOH. Stirred vigorously under H2, 2.5 hr. Filtered through celite, added 3 eq NaOMe/MeOH, 2 hr. Quenched with HCl, 95% yield (c) UMP-morpholidate (1.6 eq.) tetrazole (3.2 eq.) trioctylamine (1.0 eq.), 70 hr. Purified by RP-HPLC (Phenomonex Luna 5 μm C18 column, 5-80% MeOH over 40 minutes), 40% yield. (d) K₂CO₃ (2.0 eq.) in 3:1 MeOH:H₂O, 2 hr. added HCl to pH 7, removed MeOH, redissolved in 0.1M NaHCO₃ (e) NHS-derivative in DMF (2 eq., 1 mg/100 μL) 2 hr. Purified by RP-HPLC (Phenomonex Luna 5 um C18 column, 5-80% MeOH over 50 minutes). 5% yield over 2 steps. (f) glutaric anhydride (1.1 eq.) AcOH, 3 hr., removed AcOH under vacuum followed by EDC (1.2 eq.) and NHS (1.2 eq.), CH₂Cl₂:DMF 10:1, 2 hr., removed CH₂Cl₂ under vacuum.

### K_{D} Measurements for Fluorescent UDP-GlcNAc Probes 2 and 3

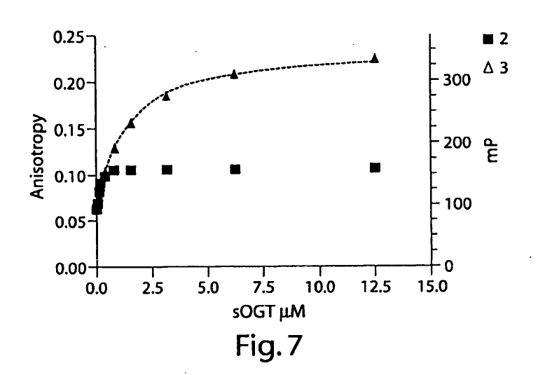

Probes 2 and 3 (also referred to herein as compounds 2 and 3, respectively) were evaluated for sOGT binding by equilibrating 48 nM probe 2 or probe 3 in 20 mM phosphate, pH=7.4, with 12.5 μM sOGT. This mixture was serially diluted into buffer containing 48 nM probe 2 or probe 3, resulting in sOGT concentrations ranging from 12.5 μM to 24 nM at a constant probe concentration. Fluorescence polarization (excitation at 480 m and emission at 535 nm) was measured using a Perkin Elmer Envision® microplate reader. Each series was performed in triplicate, and the average of each data point was converted from fluorescence polarization to fluorescence anisotropy (eq 1), then fitted (FIG. 7) to the standard equation (eq 2) describing the equilibrium L+E.LE (L=ligand, E=enzyme, LE=complex) to determine the K_{D} of the substrate analogs.

A=2P/(3−P) eq 1

Where: A=measured anisotropy, P=measured polarization:

A=Aₘᵢₙ+((L+E+KD)−((L+E+KD)2−(4LE))1/2)(Aₘₐₓ−Aₘᵢₙ)/(2L) eq 2

Where: A=measured anisotropy at a particular total concentration of sOGT (E) and probe 2 or 3 (L), Aₘᵢₙ=minimum polarization (i.e., anisotropy of free compound), Aₘₐₓ=final anisotropy (i.e., anisotropy of probe 2 or probe 3 bound entirely to sOGT), and KD=dissociation constant.

Fitting the data to this equation we calculated KD=1.3±0.1 μM for probe 3 with sOGT but anisotropy vs. sOGT with Probe 2 could not be fit using non-linear regression and was not used further. In an ideal substrate analog displacement assay, the probe is entirely bound to the enzyme at equilibrium and completely displaced in the presence of inhibitor resulting in the largest signal (maximal ΔmP) possible. The first criterion is approximated at prohibitively high protein concentrations, and a balance must always be struck between assay signal and protein consumption. In this case, a decrease in sOGT concentration from 12.5 μM to 1.5 μM resulted in a decrease of the maximal ΔmP by 100 (from ˜240 to ˜140) and results in a Z′ decrease from 0.961 to 0.854. Since it is generally accepted that a maximal ΔmP>150 and a Z′>0.8 define a robust FP assay, we chose to develop the assay at 1.5 μM sOGT. Using this enzyme concentration in a screen of 200,000 wells with a volume of 20 μL will consume ˜450 mg of sOGT.

Validation of the Displacement Assay with UDP and UDP-GlcNAc

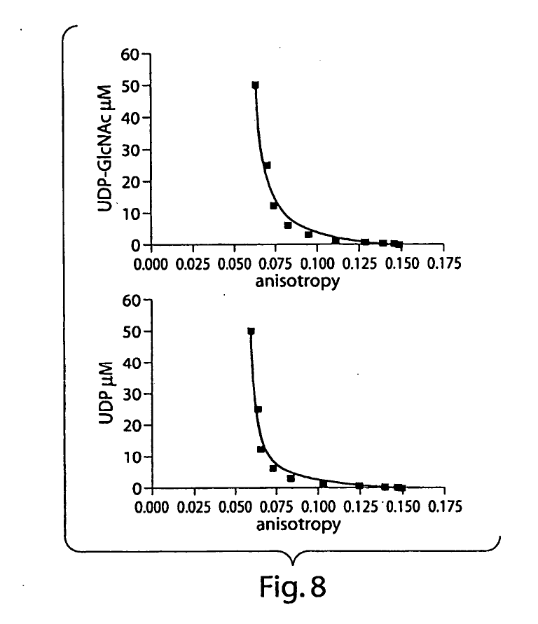

The assay protocol was to mix sOGT (1.5 μM) and probe 3 (48 nM) in 20 mM phosphate buffer, pH 8.0. 20 μL of this solution is transferred to wells of a 384-well microplate. To validate that probe 3 could be displaced from the enzyme by an inhibitor, 0.5 μL of a 4 mM UDP or UDP-GlcNAc was added to 40 μL of the pre-equilibrated probe/enzyme mixture containing 50 μM probe 3, 1.5 μM sOGT, and 20 mM phosphate buffer, pH=7.4. Serial dilutions of this mixture into buffer containing 50 nM probe 3 and 1.5 μM sOGT resulted in UDP or UDP-GlcNAc concentrations ranging from 50 μM to 98 nM (FIG. 8). Fluorescence polarization was measured using a Perkin Elmer Envision® microplate reader. Bach series was performed in triplicate and the average of each data point was converted from mP to anisotropy then fitted to the following equation:

[L]0=(KD(Aₘₐₓ−A)/Kd(A−Aₘᵢₙ)+1)([E]0−Kd(A−Aₘᵢₙ)/(Aₘₐₓ−A)−[t]0(A−Aₘᵢₙ)/(Aₘₐₓ−Aₘᵢₙ)) eq 3

where A=measured anisotropy at a particular total concentration of ligand (L), sOGT (E) and probe 3 (t), Aₘᵢₙ=minimum polarization (i.e., anisotropy of free probe), Aₘₐₓ=final anisotropy (i.e., anisotropy of probe 3 bound entirely to sOGT), K_{D}=dissociation constant of the ligand, K_{d}=dissociation constant of the probe 3.

Both UDP and UDP-GlcNAc fit the curve well and displacement occurred in less than 5 minutes. At high concentrations of both compounds, the anisotropy values were the same as for free probe 3 (FIG. 8), indicating that probe displacement can be driven to completion. A K_{D}=1.5±0.4 μM was obtained for UDP-GlcNAc and K_{D}=0.8±0.3 μM for UDP. The K_{D} of UDP-GlcNAc is similar to the K_{d} of probe 3, as expected. Moreover, both of these values are similar to the reported Kₘ and K_{d} of UDP-GlcNAc and UDP, respectively (Kreppel, L. K., & Hart, G. W. J. Biol. Chem. 1999, 274, 32015-22). We have also measured probe displacement with UDP-GlcNAc in the presence of 2% DMSO and over a 6 hour timecourse. No significant changes were observed.

### High Throughput Screening Protocol and Data Analysis

384-well plates (Costar #3654, Corning) were filled using a liquid handling robot with 20 μL of a mixture of 50 μM probe 3, 1-2 μM sOGT, and buffer (20 mM potassium phosphate, pH=7.4 with 500 μM tris(hydroxypropyl)phosphine. Compound libraries were then transferred to the assay plates using a 100 nL pin array, resulting in a final compound concentration of 25 μg/mL or ˜70 μM, assuming a average compound MW of 350. Using a Perkin Elmer Envision® microplate reader, the sample was excited at 480 nm in the vertical plane, and simultaneous emission intensity (535 nm) of the vertical and horizontal polarization planes was measured. The polarization was calculated using the following equation:

mP=1000*(V−G*H)/(V+G*H) eq 4

where; mP=millipolarization units, V=intensity of vertically polarized emission (RFU), H intensity of horizontally polarized emission (RFU), and G=gain.

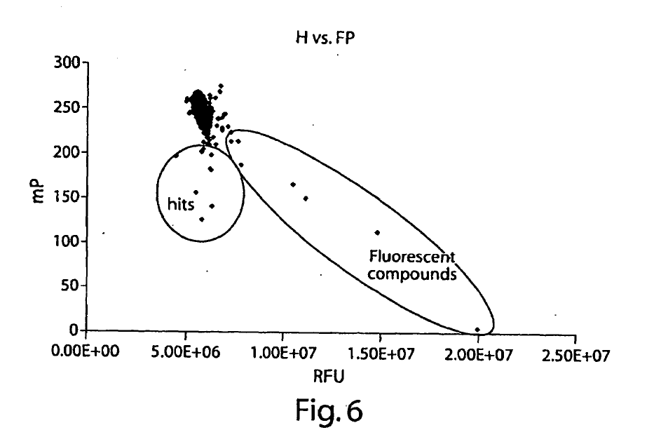

Compounds that caused a significant decrease in FP without an increase in V in both duplicate sets were scored as a hit (see FIG. 6).

The ICCB libraries BIOMOL ICCB Known Bioactives, Mixed Commercial Plate 5, Maybridge 3, Maybridge 4, I.F. Lab 1, Enamine 1, ChemDiv 2, Chemdiv 3, Biomol-TimTec 1, and Bionet 2, and a portion of ChemDiv 1 (plates 628-662) were screened over five days. Descriptions of these libraries and vendor information are available at: iccb.med.harvard.edu/screening/compound_libraries/index.htm, also see Results and Discussion section below herein for vendor information.

### Secondary Assay (Secondary Screen Protocol)

Hits were examined in a secondary assay involving the transfer of UDP-¹⁴C-GlcNAc to a peptide derived from casein kinase II, which contains a known OGT glycosylation site, as described by Hart and co-workers (Kreppel, L. K., & Hart, G. W. J. Biol. Chem. 1999, 274, 32015-22). Three lysines and a tyrosine were added to the N-terminus of this peptide substrate (KKKYPGGSTPVSSANMM) (SEQ ID NO:2) to allow rapture on phosphocellulose discs and UV detection, respectively.

One microliter aliquots (5 mg/nL) of the hits were obtained from the ICCB, and these were normalized to 1.0 mM with DMSO. These compounds were screened at 25 μM in a reaction mixture containing 500 μM peptide (SEQ ID NO:2), 6.25 μM UDP-¹⁴C-GlcNAc, ˜20-40 nM sOGT, and buffer (125 mM NaCl, 1 mM EDTA, 20 mM potassium phosphate, pH=7.4, and 500 μM tris(hydroxypropyl)phosphine. Reactions were spotted on Whatman P81 phosphocellulose discs, washed three times for five minutes in 1% phosphoric acid, and counted by liquid scintillation counting.

### Results and Discussion

The expression of OGT for structural and biochemical studies has been optimized. The yield of OGT in previously reported expression systems, including a report of expression in E. coli, did not appear to be sufficient for studies to assess OGT inhibitors so bacterial expression was optimized. Using an assembly PCR approach, a splice variant of human OGT (mOGT) was synthesized using 40-mer oligonucleotides that coded for the human protein but used optimized E. coli codons. When the gene had been obtained the expression conditions were optimized by modifying both N-terminal and C-terminal tags, making protein truncations, and by varying growth temperature and induction conditions.

Optimization led to a series of constructs that could be produced in large amounts (>10 mg/L of culture). Access to this amount of protein allowed the development a substrate analog displacement assay. Development of the assay required the synthesis of several new fluorescently labeled substrate analogs, described herein as probes 1, 2, and 3. In addition to fluorescein analogs, other dyes (e.g. BODIPY, Rhodamine) were attached to UDP-Glucosamine through an amide bond. After identification of the proper linker, assay development and miniaturization was performed. Approximately ˜70,000 compounds from commercial libraries from the following vendors were screened:
TimTec Inc., 301-A Ruthar Drive, Newark, Del. 19711;
Chendiv Inc., 11558 Sorrento Valley Rd., San Diego, Calif. 92121 USA;
ENAMINE Ltd., 23 Alexandra Matrosova Street, 01103 Kiev, Ukraine;
Life Chemicals Inc., 2477 Glenwood School Drive Suite 203, Burlington, ON, L7R 3R9, Canada;
Ryan Scientific, P.O. Box 845, Isle of Palms, S.C. 29451; and
Maybridge Ltd., Trevillett, Tintagel, Cornwall PL34 0HW, England

The assay was initially performed and the primary data analyzed as described in the Methods section, with one exception—the assay buffer used contained no tris(hydroxypropyl)phosphine (THP-a reducing agent). Many hits were obtained from this assay that generally fell into two structural classes, core structures shown below:

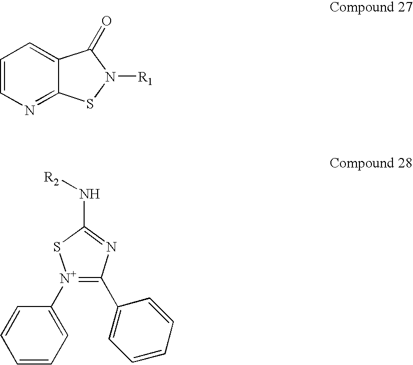

Where R₁ is an alkyl or amide and where R₂ is a hydrogen or alkyl. The complete hit set for these two cores is provided herein as compounds 15-26. With a single exception, all screened compounds in these two structural classes were hits in the assay and were also strong inhibitors (many were high nanomolar) in our secondary assay (described in the Methods section). The identified inhibitors were sulfhydryl-reactive and irreversible inhibitors of OGT, likely by reacting with a cysteine in or near the active site. Because the library contained many sulfhydryl-reactive compounds that were not hits in the assay, these compounds may have some affinity for the catalytic site of OGT and are therefore useful in designing non-reactive analogs.

The ability to obtain large amounts of protein allowed investigation of possible high-throughput screens. Ligand displacement assays in which fluorescence polarization (FP) is monitored are being used increasingly for high-throughput screening (HTS) because they are technically simple to implement if an appropriate ligand can be identified. Furthermore, they result in hits that are biased toward compounds that bind in the same location as the fluorescent probe, which can simplify the analysis of structure/activity relationships (Helm, J. S. et al., J. Am. Chem. Soc. 125: 11168-11169, 2003; Hu, Y. et al., Chem. Biol. 11: 703-711, 2004; Soltero-Higgin, M. et al., J. Am. Chem. Soc. 126: 10532-10533, 2004.). A fluorescent UDP-GlcNAc displacement assay was previously developed for a Gtf involved in peptidoglycan biosynthesis (MurG) (Helm, J. S. et al., J. Am. Chem. Soc. 125: 11168-11169, 2003), and it was determined whether a similar assay could be used to screen OGT and whether hits obtained would be selective for OGT relative to MurG.

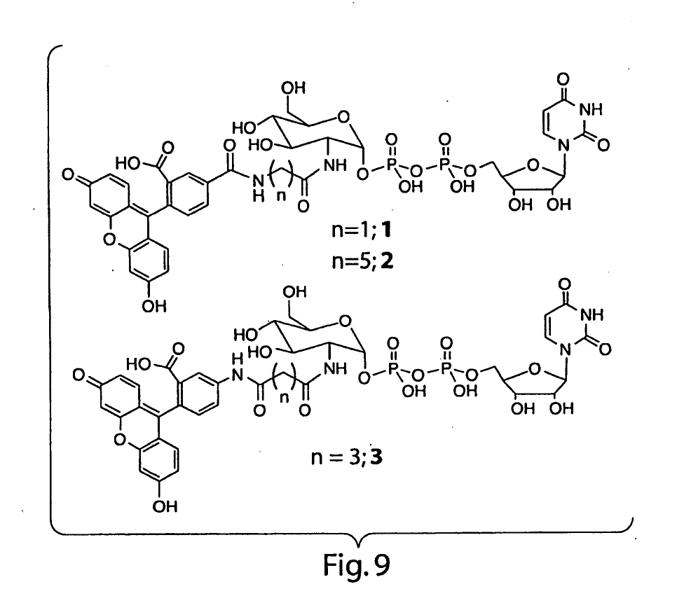

There was no structure of the Gtf domain of OGT to guide the design of a fluorescent UDP-GlcNAc analogue, but it had been proposed that OGT is structurally related to MurG (Wrabl, J. O. et al., J. Mol. Biol. 314: 365-374, 2001.). We evaluated whether the fluorescent probe used to screen MurG (probe 1, FIG. 9) could also be used in an OGT screen (Mizanur, R. M. et al., J. Am. Chem. Soc. 127: 836-837, 2005; Vocadlo, D. J. et al., Proc. Natl. Acad. Sci. USA 100: 9116-9121, 2003.). The FP of a 50 nM solution of probe 1 in the presence of increasing amounts of sOGT did not change significantly until high concentrations of protein were added. We tested the possibility that the short linker between the sugar and the fluorophore interfered with binding to the enzyme, by preparing two additional fluorescent UDP-GlcNAc analogues containing longer linkers (probe 2 and probe 3 (compounds 2 and 3), FIG. 9). Significant FP changes were observed for both probe 2 and probe 3 in the presence of sOGT, but the change for probe 3 was larger over a wider range of protein concentrations. This compound was selected as the assay probe. From the change in polarization as a function of sOGT concentration, a dissociation constant of 1.3±0.1 μM was calculated for probe 3. Addition of unlabeled UDP-GlcNAc or UDP to a pre-equilibrated mixture of sOGT and probe 3 resulted in a decrease in polarization, and both compounds completely displaced probe 3 from sOGT at high concentrations. Dissociation constants were calculated from the displacement curves and found to be 1.5±0.4 μM for UDP-GlcNAc, which implies that the fluorophore on probe 3 does not affect binding, and 0.8±0.3 μM for UDP, which agrees well with previously reported values (Haltiwanger, R. S. et al., J. Biol. Chem. 267: 9005-9013, 1992.).

The above experiments established the feasibility of a donor displacement assay for high-throughput screening (HTS) of OGT, and the assay was adapted to a 384-well microplate format and used to screen 64 416 commercial library compounds at the Institute of Chemistry and Cell Biology (ICCB) at Harvard Medical School, Boston, Mass. The libraries were screened in duplicate at a final concentration of 25 μg/mL using a Perkin-Elmer Envision microplate reader. In this screen, the assay buffer used did contain tris(hydroxypropyl)phosphine (THP-a reducing agent). Included in the compounds screened were 12 390 molecules that had previously been screened against MurG (Helm, J. S. et al., J. Am. Chem. Soc. 125: 11168-11169, 2003). This subset contained 58% of the hits that were identified in the MurG screen. Each plate contained a positive control well containing sOGT, probe 3, and 1 mM UDP-GlcNAc and a negative control well containing sOGT and probe 3. Compounds that reproducibly caused a significant decrease in FP without a corresponding change in fluorescence intensity were scored as hits. Using this criterion, 102 compounds were scored as positives, for a hit rate of 0.2%.

The positive compounds were then evaluated for OGT inhibition using a radiometric assay that involves monitoring transfer of ¹⁴C-GlcNAc to an OGT acceptor peptide containing an N-terminal (Lys)₃ tag that enables capture on phosphocellulose filter disks (Kreppel, L. K. et al., J. Biol. Chem. 274: 32015-32022, 1999; see Secondary Screen Protocol herein). Nineteen of these 102 compounds inhibited sOGT>40% at 25 μM. These molecules do not share obvious common structural features. However, this may be a consequence of library diversity since fewer than five compounds with the same core are present in the screened libraries for almost all of the 19 inhibitors. IC₅₀ values were determined for several compounds, and the mode of inhibition was determined for two of the best; both were found to be competitive with respect to UDP-GlcNAc. All of the compounds examined also inhibited the full-length construct, ncOGT.

Remarkably, none of compounds that were identified as hits in the MurG screen, which was based on displacement of UDP-GlcNAc analogue probe 1, were found to displace UDP-GlcNAc analogue probe 3 from OGT. Furthermore, none of the OGT inhibitors identified in this screen were found to inhibit MurG. Thus, there was no overlap in the compounds selected in the two high-throughput screens, even though both screens were based on displacement of the same glycosyl donor, UDP-GlcNAc, and led to the discovery of compounds that compete with this donor. There may be substantial differences in the binding pockets for UDP-GlcNAc in these enzymes that can be exploited to develop specific inhibitors. The ability to use the same screening strategy against different Gtfs could have clear advantages for the rapid discovery of orthogonal inhibitors for enzymes that use similar substrates.

We are currently investigating the effects of these compounds in cell culture. If they reduce O-GlcNAcylation in cells, they could be useful tools for probing the biological functions of OGT. In the meantime, the ability to obtain large quantities of the catalytic domain of OGT enables structural analysis of this biologically important enzyme.

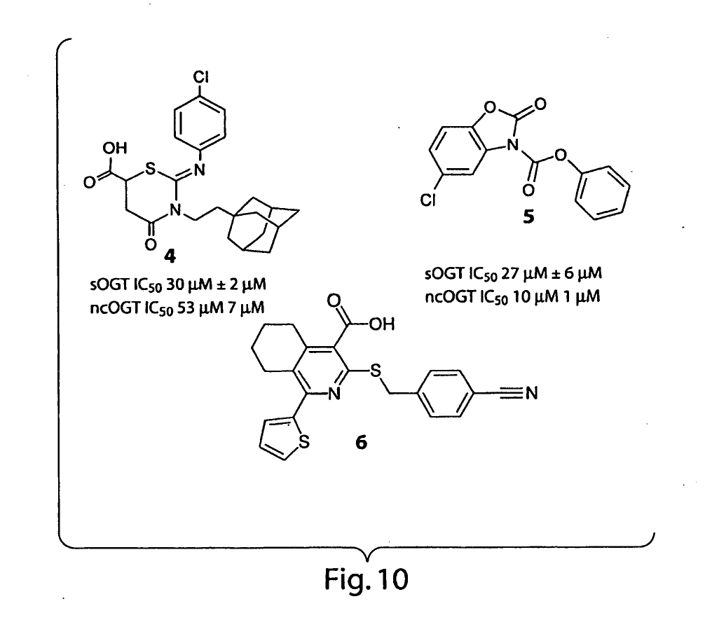

Compounds identified using the described assay in the presence of THP, included the compounds 4-6 shown in FIG. 10 and below and compounds 7-14 shown below. These compounds were identified as OGT inhibitors in the secondary assay.

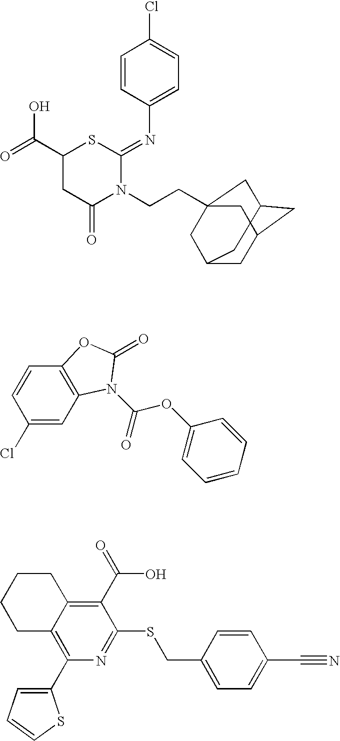

Top left=compound 4, top right=compound 5, and bottom=compound 6.

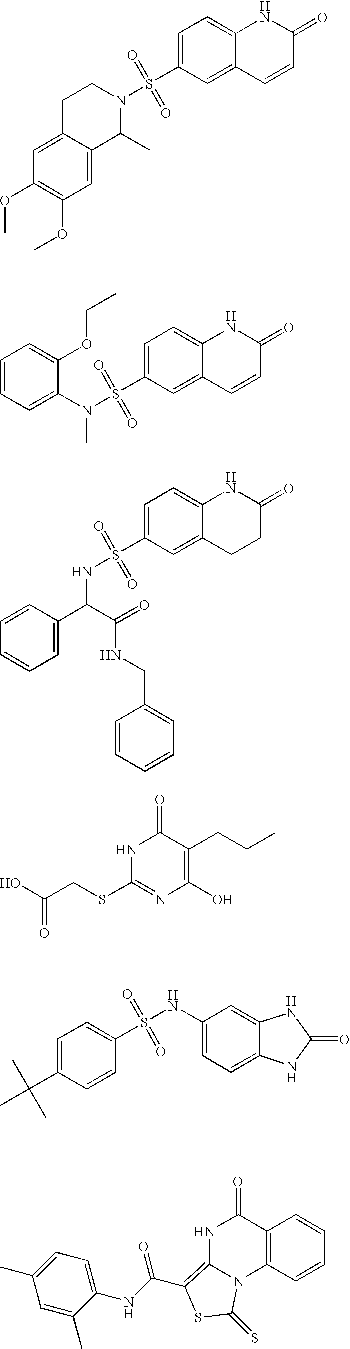

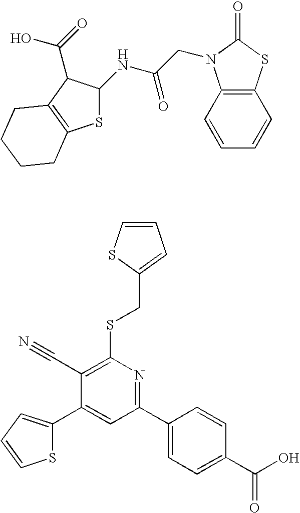

Top row: left=compound 7, center=compound 8, right=compound 9.
Middle row: left=compound 10, center=compound 11, right=compound 12.
Bottom row: left=compound 13, right=compound 14.

A subset of these compounds contains conserved features such as a 1H-quinolin-2-one or a 3,4-Dihydro-1H-quinolin-2-one core and a sulfonamide, and another compound has a similar tetra-substituted pyridine core similar to a reported compound. Additional compounds tested for use as inhibitors of OGT may be based on these core structures with substituted or additional chemical groups.

### Example 2

Compounds 4-14 have been evaluated with respect to their inhibition of OGT in cell culture. Results indicate that these compounds decrease O-GlcNAcylation in CHO cells.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. An isolated compound set forth as compound 4, or a derivative, analog, or variant thereof; compound 5, or a derivative, analog, or variant thereof; compound 6, or a derivative, analog, or variant thereof; compound 7, or a derivative, analog, or variant thereof; compound 8, or a derivative, analog, or variant thereof; compound 9, or a derivative, analog, or variant thereof; compound 10, or a derivative, analog, or variant thereof; compound 11, or a derivative, analog, or variant thereof; compound 12, or a derivative, analog, or variant thereof; compound 13, or a derivative, analog, or variant thereof; or compound 14, or a derivative, analog, or variant thereof.

2. A composition comprising an isolated compound of claim 1 and a pharmaceutically acceptable carrier.

3. (canceled)

4. A method for treating an OGT-associated disease or condition in a subject, comprising:
administering to a subject in need of such treatment an effective amount of an OGT-inhibiting compound to treat the OGT-associated disease or condition.

5. The method of claim 4, wherein the OGT-inhibiting compound is the compound set forth as compound 4 or an analog, derivative, or variant thereof that inhibits OGT activity; compound 5 or an analog, derivative, or variant thereof that inhibits OGT activity; compound 6 or an analog, derivative, or variant thereof that inhibits OGT activity; compound 7 or an analog, derivative, or variant thereof that inhibits OGT activity; compound 8 or an analog, derivative, or variant thereof that inhibits OGT activity; compound 9 or an analog, derivative, or variant thereof that inhibits OGT activity; compound 10 or an analog, derivative, or variant thereof that inhibits OGT activity; compound 11 or an analog, derivative, or variant thereof that inhibits OGT activity; compound 12 or an analog, derivative, or variant thereof that inhibits OGT activity; compound 13 or an analog, derivative, or variant thereof that inhibits OGT activity; or compound 14 or an analog, derivative, or variant thereof that inhibits OGT activity.

6.-15. (canceled)

16. The method of claim 4, wherein the subject is human.

17. The method of claim 4, wherein the OGT-inhibiting compound is linked to a targeting molecule.

18. The method of claim 4, wherein the OGT-inhibiting compound is administered prophylactically to a subject at risk of having a OGT-associated disease or disorder.

19. The method of claim 4, wherein the OGT-inhibiting compound is administered in combination with an additional drug for treating a OGT-associated disease or disorder.

20. The method of claim 4, wherein the OGT-associated disease or disorder is Alzheimer's disease; cancer; diabetes mellitus, insulin resistance, or a complication of diabetes.

21. The method of claim 20, wherein the complication of diabetes is microvascular damage, insulin resistance, vascular damage, nephropathy, skin ulcers, circulatory damage, diabetic nephropathy, diabetic retinopathy, macro-vascular disease, micro-vascular disease, or diabetic neuropathy.

22. A method of evaluating the effect of a candidate compound on OGT activity, comprising:
contacting OGT bound to a probe with a candidate compound and
determining the displacement of the probe from the OGT,
wherein the displacement of the probe from the OGT indicates that the candidate compound inhibits OGT activity.

23. The method of claim 22, wherein the probe is compound 2 or 3.

24. The method of claim 22, wherein the displacement of the probe from the OGT is determined using a method comprising detecting fluorescence polarization.

25.-41. (canceled)

42. A method for inhibiting OGT activity in a cell or tissue, comprising: contacting the cell or tissue with an effective amount of an OGT-inhibiting compound to inhibit OGT activity in the cell, or tissue.

43. The method of claim 42, wherein the OGT-inhibiting compound is the compound set forth as compound 4 or an analog, derivative, or variant thereof that inhibits OGT activity; compound 5 or an analog, derivative, or variant thereof that inhibits OGT activity; compound 6 or an analog, derivative, or variant thereof that inhibits OGT activity; compound 7 or an analog, derivative, or variant thereof that inhibits OGT activity; compound 8 or an analog, derivative, or variant thereof that inhibits OGT activity; compound 9 or an analog, derivative, or variant thereof that inhibits OGT activity; compound 10 or an analog, derivative, or variant thereof that inhibits OGT activity; compound 11 or an analog, derivative, or variant thereof that inhibits OGT activity; compound 12 or an analog, derivative, or variant thereof that inhibits OGT activity: compound 13 or an analog, derivative, or variant thereof that inhibits OGT activity; or compound 14 or an analog, derivative, or variant thereof that inhibits OGT activity.

44.-53. (canceled)

54. The method of claim 42, wherein the OGT-inhibiting compound is linked to a targeting molecule.

55. An isolated nucleic acid molecule comprising the nucleotide sequence set forth as SEQ ID NO: 1.

56. An isolated protein encoded by the nucleotide sequence set forth as SEQ ID NO:1.

57. An isolated polypeptide comprising the amino acid sequence set forth as SEQ ID NO:2.

58. A method for confirming the effect of a candidate compound as an inhibitor of OGT activity, the method comprising,
(a) contacting a sample containing the candidate compound with
(i) an OGT substrate polypeptide,
(ii) a UDP-GlcNAc probe comprising detectably labeled GlcNAc, and
(iii) OGT; and
(b) determining the amount of transfer of the labeled GlcNAc to the OGT substrate polypeptide in the sample, wherein a lower amount of GlcNAc transferred to the OGT substrate in the sample compared to a control amount of GlcNAc transfer to the OGT substrate confirms the effect of the candidate compound as an inhibitor of OGT activity.

59.-68. (canceled)
